(19) United States
(12) Patent Application Publication
(10) Pub. No.: US 2010/0222219 A1
(43) Pub. Date: Sep. 2, 2010

(54) Thiophene-Sulphonic Acid Picolyl Amides

(75) Inventors: Jan Klaas Lohmann, Ludwigshafen (DE); Wassilios Grammenos, Ludwigshafen (DE); Michael Puhl, Lampertheim (DE); Jochen Dietz, Mannheim (DE); Bernd Müller, Frankenthal (DE); Joachim Rheinheimer, Ludwigshafen (DE); Jens Renner, Bad Durkheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/438,169

(22) PCT Filed: Aug. 13, 2007
(86) PCT No.: PCT/EP2007/058348

§ 371 Date: Feb. 20, 2009

(30) Foreign Application Priority Data
Aug. 22, 2006 (EP) 06119331.4

(51) Int. Cl. A01C 1/06; C07D 409/12; A01N 43/40; A01P 7/00; A01P 7/04

(52) U.S. Cl. 504/100; 546/280.4; 514/336

(57) ABSTRACT

The present invention relates to thiophene-sulfonic acid picolyl amides of the Formula (I), where R^{I} to R⁷ and n are as defined in the claims and to the N-oxides, the agriculturally acceptable salts and the veterinarily acceptable salts of the compounds (I), with the proviso that if the thiophene ring is bonded to the sulfonyl group via position 2, R⁶ cannot be at position 5. The invention also relates to a process for preparing these compounds. Furthermore, the invention relates to the use of the compounds I and the N-oxides and the agriculturally acceptable salts thereof for combating phytopathogenic fungi (hereinafter referred to as harmful fungi). Additionally, the compounds (I), their N-oxides and salts can be used for controlling arthropodal pests. Furthermore, the invention relates to seed comprising a compound (I) or an N-oxide or agriculturally acceptable salt thereof.

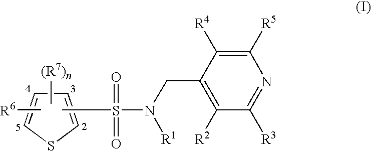

The present invention relates to thiophene-sulfonic acid picolyl amides of the formula I

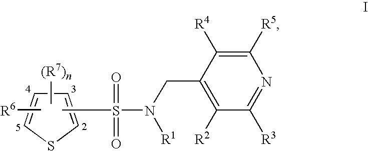

where
R¹ is hydrogen, C₁-C₄-alkyl, C₁-C₄-alkoxy, cyano-C₁-C₄-alkyl, C₁-C₄-haloalkyl, C₁-C₄-alkoxy-C₁-C₄-alkyl, C₁-C₄-haloalkoxy-C₁-C₄-alkyl, di(C₁-C₄-alkyl)amino-C₁-C₄-alkyl, C₃-C₆-cycloalkyl-C₁-C₄-alkyl, C₃-C₆-halocycloalkyl-C₁-C₄-alkyl, (C₁-C₄-alkyl)-carbonyl, (C₁-C₄-alkoxy)carbonyl, C₂-C₄-alkenyl, cyano-C₂-C₄-alkenyl, C₂-C₄-halo-alkenyl, C₁-C₄-alkoxy-C₂-C₄-alkenyl, C₁-C₄-haloalkoxy-C₂-C₄-alkenyl, (C₁-C₄-alkyl)carbonyl-C₂-C₄-alkenyl, (C₁-C₄-alkoxy)carbonyl-C₂-C₄-alkenyl, di(C₁-C₄-alkyl)amino-C₂-C₄-alkenyl, C₂-C₄-alkynyl, C₂-C₄-haloalkynyl, C₁-C₄-alkyl-C₂-C₄-alkynyl, C₁-C₄-haloalkyl-C₂-C₄-alkynyl, C₁-C₄-alkoxy-C₂-C₄-alkynyl, di(C₁-C₄-alkyl)amino, or benzyl which may bear at the phenyl ring a cyano, halogen, C₁-C₄-alkyl, C₁-C₄-haloalkyl, C₁-C₄-alkoxy, C₁-C₄-haloalkoxy, (C₁-C₄-alkyl)-carbonyl, (C₁-C₄-alkoxy)carbonyl or di(C₁-C₄-alkyl)amino radical;
R², R³ independently of one another are selected from hydrogen, thiol, amino, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkyl-sulfinyl, C₁-C₄-alkylsulfonyl, C₁-C₄-haloalkylsulfonyl, (C₁-C₄-alkyl)amino, di(C₁-C₄-alkyl)amino, tri-C₁-C₄-alkylsilyl, C₂-C₄-alkenyl, C₂-C₄-alkynyl or a 5- or 6-membered heterocyclyl ring containing one nitrogen atom and optionally a second heteroatom selected from oxygen, sulfur, NH or N(C₁-C₄-alkyl);
R⁴, R⁵ independently of one another are selected from hydrogen, halogen, C₁-C₄-alkyl, C₁-C₄-haloalkyl, C₁-C₄-alkoxy or C₁-C₄-haloalkoxy; or
   R² and R³ together with the carbon atoms to which they are attached, may form a condensed phenyl, cyclopentyl, cyclohexyl or 5- or 6-membered heterocyclyl ring containing one to three heteroatoms selected from the group consisting of 2 nitrogen, 1 oxygen and 1 sulfur atoms,
   it being possible for all these rings to carry one or two groups R⁸ and/or R⁹,
R⁸, R⁹ independently of one another are halogen, C₁-C₄-alkyl, C₁-C₄-haloalkyl, C₁-C₄-alkoxy or C₁-C₄-haloalkoxy;
R⁶ is halogen, cyano, nitro, C₁-C₆-alkyl, C₁-C₆-alkoxy, C₁-C₆-haloalkoxy, (C₁-C₄-alkyl)carbonyl, (C₁-C₄-alkoxy)carbonyl, —C(R¹⁰)═NOR¹¹,
   (C₁-C₄-alkyl)aminocarbonyl, di(C₁-C₄-alkyl)aminocarbonyl,
   phenyl or phenoxy, where the ring in the last two mentioned radicals may carry one, two or three groups R¹²;
R⁷ cyano, formyl, halogen, C₁-C₄-alkyl, C₁-C₄-haloalkyl, C₁-C₄-alkoxy, C₁-C₄-haloalkoxy, C₁-C₄-alkoxy-C₁-C₄-alkyl, (C₁-C₄-alkoxy)carbonyl, aminocarbonyl, C₁-C₄-alkylaminocarbonyl or di(C₁-C₄-alkyl)aminocarbonyl;
n is zero or one;
or
R⁶ and R⁷ together with the carbon atoms to which they are attached, may form a condensed phenyl ring, it being possible for the phenyl ring to carry one C₁-C₄-alkyl group;
R¹⁰ is hydrogen, C₁-C₄-alkyl, C₁-C₄-haloalkyl, C₁-C₄-alkoxy-C₁-C₄-alkyl, C₁-C₄-haloalkoxy-C₁-C₄-alkyl, phenyl which may bear a cyano, halogen, C₁-C₄-alkoxy or C₁-C₄-haloalkoxy radical, or
   benzyl which may bear a cyano, halogen or C₁-C₄-alkyl radical;
R¹¹ is C₁-C₆-alkyl, benzyl, C₂-C₄-alkenyl, C₁-C₄-haloalkyl, C₂-C₄-haloalkenyl, C₂-C₄-alkynyl or C₂-C₄-haloalkynyl;
R¹² is nitro, cyano, halogen, C₁-C₄-alkyl, C₁-C₄-haloalkyl, C₁-C₄-alkoxy, C₁-C₄-haloalkoxy, (C₁-C₄-alkoxy)carbonyl, C₁-C₄-alkylthio, C₁-C₄-haloalkylthio, C₁-C₄-alkylsulfonyl, C₁-C₄-haloalkylsulfonyl, (C₁-C₄-alkyl)amino, di(C₁-C₄-alkyl)amino, tri(C₁-C₄-alkyl)silyl, —CH═NO(C₁-C₄-alkyl), —C(C₁-C₄-alkyl)═NO(C₁-C₄-alkyl), C₂-C₄-alkenyl or C₃-C₄-alkinyl;
   or two radicals R¹² may form a C₃-C₄-alkylene or C₄-alkenylene chain which, together with two adjacent ring members of the aryl ring to which it is attached, forms a ring which may be substituted by one to 3 groups R¹³;
R¹³ is halogen, cyano, nitro, C₁-C₈-alkyl, C₁-haloalkyl, C₁-C₈-alkoxy, C₁-C₈-haloalkoxy, (C₁-C₄-alkyl)carbonyl, (C₁-C₄-alkoxy)carbonyl, —C(R¹⁴)═NOR¹⁵, (C₁-C₄-alkyl)aminocarbonyl, di(C₁-C₄-alkyl)aminocarbonyl or
   phenyl or phenoxy, where the ring in the last two mentioned radicals may carry one, two or three groups R¹⁶;
R¹⁴ is hydrogen, C₁-C₄-alkyl, C₁-C₄-haloalkyl, C₁-C₄-alkoxy-C₁-C₄-alkyl, C₁-C₄-haloalkoxy-C₁-C₄-alkyl, phenyl which may bear a cyano, halogen, C₁-C₄-alkoxy or C₁-C₄-haloalkoxy radical, or
   benzyl which may bear a cyano, halogen or C₁-C₄-alkyl radical;
R¹⁵ is C₁-C₆-alkyl, benzyl, C₂-C₄-alkenyl, C₁-C₄-haloalkyl, C₂-C₄-haloalkenyl, C₂-C₄-alkynyl or C₂-C₄-haloalkynyl;
R¹⁶ is halogen, C₁-C₄-alkyl, C₁-C₄-alkoxy, C₁-haloalkyl or C₁-haloalkoxy;
   and the N-oxides and the agriculturally acceptable salts and the veterinarily acceptable salts of the compounds I,
   with the proviso that if the thiophene ring is bonded to the sulfonyl group via position 2, R⁶ cannot be at position 5.

Moreover, the invention relates to processes for preparing these compounds, to compositions comprising them and to their use for controlling phytopathogenic harmful fungi.

The present invention also relates to the use of the thiophene-sulfonic acid picolyl amides I for combating arthropodal pests (harmful arthropodes) and for protecting crops, seed and materials against infestation and/or destruction by said pests.

WO 2005/33081 describes 4-pyridylmethyl amides of benzenesulfonic acid compounds and their use for combating harmful fungi. However, the action of the compounds disclosed there is not always completely satisfying. Therefore, it was an object of the present invention to provide compounds having improved action and/or a broadened activity spectrum against harmful fungi.

It was found that this object is achieved by the compounds of the formula I, their N-oxides and salts, as defined herein. Compared to the known compounds, the compounds of the formula I have increased efficacy against harmful fungi. Therefore the invention relates to compounds of the general formula I, their N-oxides and the salts thereof. The invention also relates to a process for preparing these compounds.

Furthermore, the invention relates to the use of the compounds I and the N-oxides and the agriculturally acceptable salts thereof for combating phytopathogenic fungi (hereinafter referred to as harmful fungi). Accordingly, the invention also provides a process for combating phytopathogenic fungi which process comprises treating the fungi or the materials, plants, the soil or seeds to be protected against fungal attack, with an effective amount of a thiophene-sulfonic acid picolyl amide of the formula I or an N-oxide or an agriculturally acceptable salt thereof.

Accordingly, the invention further provides agricultural compositions, preferably in the form of directly sprayable solutions, emulsions, pastes, oil dispersions, powders, materials for scattering, dusts or in the form of granules, which comprises a pesticidally effective amount of at least one compound I, an N-oxide or a salt thereof, and at least one carrier which may be liquid and/or solid and which is preferably agronomically acceptable, and/or at least one surfactant.

Furthermore, it was found that the compounds I, their N-oxides and salts can be used for controlling arthropodal pests. The compounds I, their N-oxides and salts are in particular useful for combating insects. Likewise the compounds I, their N-oxides and salts are in particular useful for combating arachnids. The term “combating arthropodal pest” as used herein comprises controlling, i.e. killing, of said pests and also protecting plants, non-living materials or seed from an attack or infestation by said pests. Therefore the invention relates to the use of the compounds I and the N-oxides and the agriculturally acceptable salts thereof, for combating arthropodal pests.

Furthermore, the invention provides a method for combating such pests, which comprises contacting said pests, their habitat, breeding ground, food supply, plant, seed, soil, area, material or environment in which the animal pests are growing or may grow, or the materials, plants, seeds, soils, surfaces or spaces to be protected from an attack of or infestation by said pest, with a pesticidally effective amount of a compound I, an N-oxide or salt thereof, as defined herein.

The invention provides in particular a method for protecting crops, including seeds, from attack or infestation by arthropodal pests and/or infection by phytopathogenic fungi, said method comprises contacting a crop with an effective amount of at least one compound of formula I or the N-oxide or salt thereof, as defined herein. The invention also provides seeds, comprising a thiophene-sulfonic acid picolyl amide of the formula I, or an N-oxide or an agriculturally acceptable salt thereof, preferably in an amount of from 0.1 g to 10 kg per 100 kg of seed.

The invention also provides a method for protecting non-living materials from attack or infestation by the aforementioned pests and/or harmful fungi, which method comprises contacting the non-living material with a effective amount of at least one compound of formula I as defined herein, with an N-oxide thereof or with a salt thereof.

Suitable compounds of formula I encompass all possible stereoisomers (cis/trans isomers, enantiomers) which may occur and mixtures thereof. Stereoisomeric centers are e.g. the carbon and nitrogen atom of the —C(R¹⁰)═NOR¹¹ and —C(R¹⁴)═NOR¹⁵ moieties, as well as asymmetric carbon atoms in the radicals R¹, R², R³, R⁴, R⁵ and/or R⁶ etc. The present invention provides both the pure enantiomes or diastereomers or mixtures thereof, the pure cis- and trans-isomers and the mixtures thereof. The compounds of the general formula I may also exist in the form of different tautomers. The invention comprises the single tautomers, if separable, as well as the tautomer mixtures. The present invention includes both the (R)- and (S)-isomers of compounds of the formula I having chiral centers as well as mixtures thereof, in particular the racemates thereof.

The compounds of the formula I can be present in different crystal modifications whose biological activity may differ. They also form part of the subject matter of the present invention.

Salts of the compounds of the formula I and of the N-oxides of formula I are preferably agriculturally acceptable or veterinarily acceptable salts. They can be formed by customary methods, e.g. by reacting the compound I with an acid of the anion in question if the compound of formula I has a basic functionality or by reacting an acidic compound of formula I with a suitable base.

Suitable agriculturally useful salts include the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄_{⁺}) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of formula I with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

halogen: fluorine, chlorine, bromine and iodine;

alkyl and all alkyl moieties in alkylcarbonyl, alkylamino, dialkylamino, tri(alkyl)silyl, dialkylaminocarbonyl, alkylsulfinyl, alkylsulfonyl: saturated straight-chain or branched hydrocarbon radicals having 1 to 4, 6, 8 or 10 carbon atoms, preferably 1 to 6 carbon atoms (C₁-C₆-alkyl), especially 1 to 4 carbon atoms (C₁-C₄-alkyl) such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; alkyl having 1 to 10 carbon atoms (C₁-C₁₀-alkyl): C₁-C₆-alkyl as mentioned above, and also, for example heptyl, octyl, 2-ethylhexyl, 2,4,4-trimethylpentyl, 1,1,3,3-tetramethylbutyl, n-nonyl and n-decyl;

alkoxy: saturated straight-chain or branched hydrocarbon radicals having 1 to 4, 6, 8 or 10 carbon atoms, preferably 1 to 6 carbon atoms, especially 1 to 4 carbon atoms, as defined herein, which is attached to the remainder of the molecule via an oxygen linkage;

haloalkyl: straight-chain or branched alkyl groups having 1 to 2, 4, 6, 8 or 10 carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

haloalkoxy and all haloalkoxy moieties in haloalkoxyalkyl, haloalkoxyalkenyl: straight-chain or branched alkyl groups having 1 to 4, 6, 8 or 10 carbon atoms, in particular 1 to 6 carbon atoms (C₁-C₆-haloalkyl), especially 1 to 4 carbon atom (C₁-C₄-haloalkyl), as mentioned above bonded through oxygen linkage, at any bond in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, 5-fluoropentoxy, 5-chloropentoxy, 5-bromopentoxy, 5-iodopentoxy, 6-fluorohexoxy, 6-chlorohexoxy, 6-bromohexoxy or 6-iodohexoxy and the like;

alkylthio: saturated straight-chain or branched hydrocarbon radicals having 1 to 4, 6, 8 or 10 carbon atoms, preferably 1 to 6 carbon atoms, especially 1 to 4 carbon atoms, as defined herein, which is attached to the remainder of the molecule via a sulfur linkage;

haloalkylthio: straight-chain or branched alkyl group having 1 to 4 carbon atoms, as mentioned above which is attached to the remainder of the molecule via a sulfur linkage, where some or all of the hydrogen atoms may be replaced by halogen atoms as mentioned above;

haloalkylsulfinyl: straight-chain or branched alkyl group having 1 to 4 carbon atoms, as mentioned above which is attached to the remainder of the molecule via an SO₂ group, where some or all of the hydrogen atoms may be replaced by halogen atoms as mentioned above;

haloalkylsulfonyl: straight-chain or branched alkyl group having 1 to 4 carbon atoms, as mentioned above which is attached to the remainder of the molecule via an SO₂ group, where some or all of the hydrogen atoms may be replaced by halogen atoms as mentioned above;

alkenyl: unsaturated straight-chain or branched hydrocarbon radicals having 2 to 4, 6, 8 or 10 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

haloalkenyl: unsaturated straight-chain or branched hydrocarbon radicals having 2 to 4 carbon atoms and one or two double bonds in any position (as mentioned above), where in these groups some or all of the hydrogen atoms may be replaced by halogen atoms as mentioned above, in particular by fluorine, chlorine and bromine;

alkynyl: straight-chain or branched hydrocarbon groups having 2 to 4, 6, 8 or 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

haloalkynyl: unsaturated straight-chain or branched hydrocarbon radicals having 2 to 4 carbon atoms and one triple bond in any position (as mentioned above), where in these groups some or all of the hydrogen atoms may be replaced by halogen atoms as mentioned above, in particular by fluorine, chlorine and bromine;

cycloalkyl: mono- or bicyclic saturated hydrocarbon groups having 3 to 6 carbon ring members, for example C₃-C₆-cycloalkyl such as cyclopropyl, cyclobutyl, cyclopentyl, and cyclohexyl;

tri(C₁-C₄-alkyl)silyl: silicium radical caring 3 C₁-C₄-alkyl groups, which may be identical or different, examples including trimethylsilyl, triethylsilyl, dimethylethylsilyl, dimethylisopropylsilyl, dimethyl-n-butylsilyl, dimethyl-2-butylsilyl, etc.

the terms “cyano-C₁-C₄-alkyl”, “C₁-C₄-alkoxy-C₁-C₄-alkyl”, “C₁-C₄-haloalkoxy-C₁-C₄-alkyl”, “di(C₁-C₄-alkyl)amino-C₁-C₄-alkyl”, “C₃-C₆-cycloalkyl-C₁-C₄-alkyl”, “C₃-C₆-halocycloalkyl-C₁-C₄-alkyl”, “saturated 5 or 6-membered N-heterocyclyl-C₁-C₄-alkyl”, as used herein, refer to C₁-C₄-alkyl, as defined herein, which is substituted by one radical selected from cyano, C₁-C₄-alkoxy, C₁-C₄-haloalkoxy, di(C₁-C₄-alkyl)amino, C₃-C₆-cycloalkyl, C₃-C₆-halocycloalkyl, saturated 5 or 6-membered N-heterocyclyl;

the terms “cyano-C₂-C₄-alkenyl”, “C₁-C₄-alkoxy-C₂-C₄-alkenyl”, “C₁-C₄-haloalkoxy-C₂-C₄-alkenyl”, “(C₁-C₄-alkyl)carbonyl-C₂-C₄-alkenyl”, “(C₁-C₄-alkoxy)-carbonyl-C₂-C₄-alkenyl”, “di(C₁-C₁-alkyl)amino-C₂-C₄-alkenyl” refer to C₂-C₄-alkenyl, as defined herein, which is substituted by one radical selected from cyano, C₁-C₄-alkoxy, C₁-C₄-haloalkoxy, (C₁-C₄-alkyl)carbonyl, (C₁-C₄-alkoxy)carbonyl, di(C₁-C₄-alkyl)amino;

the terms “C₁-C₄-haloalkyl-C₂-C₄-alkynyl”, “C₁-C₄-alkoxy-C₂-C₄-alkynyl”, “tri(C₁-C₄-alkyl)silyl-C₂-C₄-alkynyl” refer to C₂-C₄-alkynyl, as defined herein, which is substituted by one radical selected from C₁-C₄-haloalkyl, C₁-C₄-alkoxy, tri(C₁-C₄-alkyl)silyl;

five- or six-memberd heterocycle which contains one, two, three or four heteroatoms from the group consisting of O, N and S, is to be understood as meaning both saturated, partially unsaturated and aromatic heterocycles having 5 or 6 ring atoms, including:
5- or 6-membered heterocyclyl which contains one, two or three nitrogen atoms and/or one oxygen or sulfur atom or one or two oxygen and/or sulfur atoms, and which is saturated or partially unsaturated, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered aromatic heterocyclyl (heteroaryl) which contains one, two, three or four nitrogen atoms or one, two or three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-thienyl, 3-thienyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl which contains one, two, three or four nitrogen atoms: 6-membered heteroaryl groups which, in addition to carbon atoms, may contain one, two, three or four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

Likewise a five- to eight-membered saturated heterocycle which is attached via nitrogen and may contain one, two or three further heteroatoms or heteroatom groups from the group consisting of O, N, S, S(O) and S(O)₂ as ring members, is a saturated heterocycle, which contains a nitrogen atom as ring member and which is bound to the remainder of the molecule via said nitrogen atom, and which has 5, 6, 7 or 8 ring atoms which are carbon atoms or heteroatoms such as O, N or S or heteroatom groups such as S(O) or S(O)₂; examples including pyrrolidin-1-yl, piperazin-1-yl, morpholin-4-yl, thiomorpholin-4-yl, azepan-1-yl etc.

Fused 5- or 6-membered carbocycle means a hydrocarbon ring which shares two adjacent carbon atoms with another ring, examples being cyclopentane, cyclopentene, cyclohexane, cyclohexene and benzene.

Examples for 5- or 6-membered heterocycles which contain a contain a fused 5 or 6 membered carbocyclic ring as mentioned above are indolyl, indolinyl, isoindolinyl, benzpyrazolyl, benzimidazolyl, benzotriazolyl, quinolinyl, 1,2,3,4-tetrahydroquinolinyl, isoquinolinyl, phthalazinyl, quinazinyl, quinazolinyl, cinnolinyl, benzofuranyl, benzothiophenyl, benzopyranyl, dihydrobenzopyranyl, benzothiopyranyl, 1,3-benzodioxolyl, benzoxazolyl, benzthiazolyl, benzisoxazolyl and 1,4-benzodioxanyl.

With a view to the intended uses of the thiophene-sulfonic acid picolyl amides I, particular preference is given to the following meanings of the substituents, in each case on their own or in combination:

The invention preferably provides compounds of the formula I in which R¹ is hydrogen, C₁-C₄-alkyl, allyl, propargyl or benzyl, in particular hydrogen.

Preference is also given to compounds of the formula I in which R², R³, R⁴ and R⁵ independently of one another are hydrogen, C₁-C₄-alkyl such as methyl or ethyl, halogen such as fluorine or chlorine, C₁-C₂-haloalkyl such as CF₃, or C₁-C₂-haloalkoxy such as OCF₃ or OCHF₂.

Particular preference is given to compounds wherein R², R³, R⁴ and R⁵ are hydrogen.

In addition, particular preference is also given to compounds of the formula I in which at least one, in particular one or two, group(s) selected from R², R³, R⁴ and R⁵ is/are not hydrogen. Amongst these, preference is given to those compounds, wherein both R⁴ and R⁵ are hydrogen, while at least one of the radicals R², R³ is different from hydrogen and has one of the meanings given above. In particular the R² and/or R³ that is different from hydrogen is selected from C₁-C₄-alkyl such as methyl or ethyl, halogen, such as fluorine or chlorine, C₁-C₂-haloalkyl such as CF₃, or C₁-C₂-haloalkoxy such as OCF₃ or OCHF₂. In this embodiment preference is also given to compounds, wherein one of the radicals R² and/or R³ is selected from amino, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, (C₁-C₄-alkyl)amino, di(C₁-C₄-alkyl)amino, tri(C₁-C₄)alkylsilyl, C₂-C₄-alkenyl or C₂-C₄-alkynyl. The remaining radical R² or R³ is preferably hydrogen or selected from the group consisting of C₁-C₄-alkyl such as methyl or ethyl, halogen, such as fluorine or chlorine, C₁-C₂-haloalkyl such as CF₃, or C₁-C₂-haloalkoxy such as OCF₃ or OCHF₂.

Preference is likewise also given to compounds of the formula I, wherein the radicals R² and R³ together with the atoms to which they are bound form a fused benzene ring, i.e. R² and R³ together form a bivalent radical —CH═CH—CH═CH—, wherein one or two of the hydrogen atoms may be replaced by the radicals R⁸ and/or R⁹. In this embodiment, R⁴ and R⁵ are preferably hydrogen.

In a first preferred embodiment, R⁶ is selected from halogen, in particular chlorine and fluorine; C₁-C₄-alkyl, in particular methyl and ethyl; C₁-C₄-alkoxy, in particular methoxy and ethoxy; C₁-C₄-haloalkyl, in particular trifluoromethyl; C₁-C₄-haloalkoxy, in particular difluoromethoxy and trifluoromethoxy; (C₁-C₄-alkoxy)carbonyl, in particular methoxycarbonyl and ethoxycarbonyl.

In a second preferred embodiment R⁶ is phenyl, which is unsubstituted or preferably carries 1, 2 or 3 radicals R¹² as defined above. More preference is given to compounds wherein one of the radicals is phenyl, which is unsubstituted or which preferably carries 1, 2 or 3 radicals R¹² as defined above. If present, the further radical R⁶ is preferably different from phenyl or phenoxy, and more preferably selected from halogen, in particular chlorine and fluorine; C₁-C₄-alkyl, in particular methyl and ethyl; C₁-C₄-alkoxy, in particular methoxy and ethoxy; C₁-C₄-haloalkyl, in particular trifluoromethyl; C₁-C₄-haloalkoxy, in particular difluoromethoxy and trifluoromethoxy; (C₁-C₄-alkoxy)carbonyl, in particular methoxycarbonyl and ethoxycarbonyl.

In one embodiment of the compounds I according to the present invention, the index n is zero.

In another embodiment of the compounds I according to the present invention, the index n is 1.

In the compounds of the formula I, the thiophene ring at the sulfonyl group may be bound via the carbon atom in the 2- or 3-position.

Consequently, one embodiment of the invention relates to compounds of formula Ia

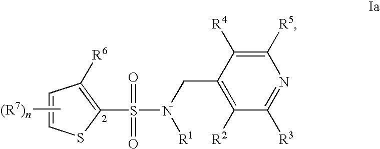

wherein R¹, R², R³, R⁴, R⁵, R⁶, R⁷ and n are as defined herein, and their N-oxides and salts. Amongst compounds Ia, preference is given to those, wherein n is zero. In the compounds Ia, R⁶ is preferably phenyl, which is unsubstituted or substituted as defined above.

Consequently, a further embodiment of the invention relates to compounds of formula Ib

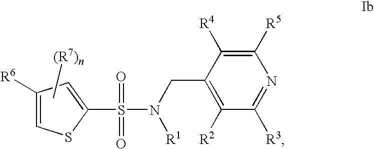

wherein R¹, R², R³, R⁴, R⁵, R⁶, R⁷ and n are as defined herein, and their N-oxides and salts. Amongst compounds Ib, preference is given to those, wherein n is zero. In the compounds Ib, R⁶ is preferably phenyl, which is unsubstituted or substituted as defined above.

Consequently, a further embodiment of the invention relates to compounds of formula Ic

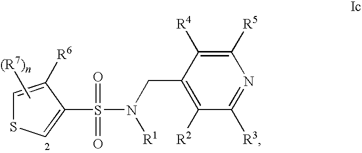

wherein R¹, R², R³, R⁴, R⁵, R⁶, R⁷ and n are as defined herein, and their N-oxides and salts. Amongst compounds Ic, preference is given to those, wherein n is zero. In the compounds Ic, R⁶ is preferably phenyl, which is unsubstituted or substituted as defined above.

Consequently, a further embodiment of the invention relates to compounds of formula Id

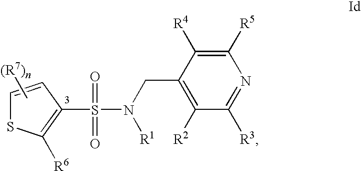

wherein R¹, R², R³, R⁴, R⁵, R⁶, R⁷ and n are as defined herein, and their N-oxides and salts. Amongst compounds Id, preference is given to those, wherein n is zero. In the compounds Id, R⁶ is preferably phenyl, which is unsubstituted or substituted as defined above.

Consequently, a further embodiment of the invention relates to compounds of formula Ie

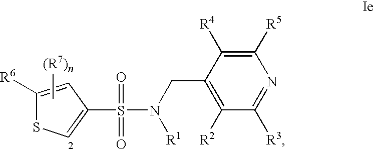

wherein R¹, R², R³, R⁴, R⁵, R⁶, R⁷ and n are as defined herein, and their N-oxides and salts. Amongst compounds Ie, preference is given to those, wherein n is zero. In the compounds Ie, R⁶ is preferably phenyl, which is unsubstituted or substituted as defined above.

R⁸, if present, is preferably selected from halogen, in particular chlorine and fluorine; C₁-C₄-alkyl, in particular methyl, ethyl, isopropyl, tert.-butyl; C₁-C₄-alkoxy, in particular methoxy, ethoxy, isopropoxy, tert.-butoxy; and C₁-C₄-haloalkyl, in particular trifluoromethyl and pentafluoroethyl.

R⁹, if present, is preferably selected from halogen, in particular chlorine and fluorine; C₁-C₄-alkyl, in particular methyl, ethyl, isopropyl, tert.-butyl; C₁-C₄-alkoxy, in particular methoxy, ethoxy, isopropoxy, tert.-butoxy; and C₁-C₄-haloalkyl, in particular trifluoromethyl and pentafluoroethyl.

R¹⁰, R¹⁴, if present, are independently of each other preferably selected from hydrogen or C₁-C₄-alkyl, in particular hydrogen.

R¹¹, R¹⁵, if present, are independently of each other preferably C₁-C₄-alkyl.

R¹², if present, is preferably selected from nitro, CN, halogen, C₁-C₄-alkyl, C₁-C₄-haloalkyl, C₁-C₄-alkoxy, C₁-C₄-haloalkoxy, (C₁-C₄-alkoxy)carbonyl, C₁-C₄-alkylcarbonyl, C₁-C₄-alkylthio, C₁-C₄-haloalkylthio, C₁-C₄-alkylsulfonyl, C₁-C₄-haloalkylsulfonyl, (C₁-C₄-alkyl)amino, di(C₁-C₄-alkyl)amino, tri(C₁-C₄-alkyl)silyl, —CH═NO(C₁-C₄-alkyl), —C(C₁-C₄-alkyl)═NO(C₁-C₄-alkyl), C₂-C₄-alkenyl or C₃-C₄-alkinyl, or two radicals R¹² together with two adjacent carbon atoms of the phenyl ring may form a radical of the formulae: (CH₂)₃, (CH₂)₄, O—CH₂—O, O(CH₂)₃ or —CH═CH—CH═CH—. R¹², if present, is more preferably selected from cyano, halogen, in particular fluorine or chlorine, C₁-C₄-alkyl, in particular methyl, ethyl, n-propyl, isopropyl or tert.-butyl, C₁-C₄-haloalkyl, in particular trifluoromethyl, difluoromethyl or trifluoroethyl, C₁-C₄-alkoxy, in particular methoxy, C₁-C₄-haloalkoxy, in particular trifluoromethoxy, C₁-C₄-alkylcarbonyl, in particular acetyl, CONH₂, —CH═NOCH₃, —C(CH₃)═NOCH₃, —CH═NOCH₂CH₃, or —C(CH₃)═NOCH₂CH₃.

R¹⁶, if present, is preferably selected from methyl, ethyl, trifluoromethyl, 2-fluoroethyl, 2,2-difluoroethyl or 2,2,2-trifluoroethyl.

Most preferably R⁶ is phenyl which carries one, two or three radicals R¹² as defined herein, in particular as given in the lines of table A. In table A, the prefix indicates the position of the phenyl ring, to which the radical R¹² is bound.

Examples of preferred compounds are given in the following tables:

### Table 1

Compounds of the formula IaA, in which R¹, R², R³, R⁴ and R⁵ are hydrogen, n is zero and R⁶ is a phenyl ring, which carries 1 or 2 radicals R¹² as defined in the rows of Table A;

### Table 2

Compounds of the formula IaB, in which R¹, R², R⁴ and R⁵ are hydrogen, R³ is chlorine, n is zero and R⁶ is a phenyl ring, which carries 1 or 2 radicals R¹² as defined in the rows of Table A;

### Table 3

Compounds of the formula IaC, in which R¹, R³, R⁴ and R⁵ are hydrogen, R² is chlorine, n is zero and R⁶ is a phenyl ring, which carries 1 or 2 radicals R¹² as defined in the rows of Table A;

### Table 4

Compounds of the formula IaD, in which R¹, R⁴ and R⁵ are hydrogen, R² is chlorine, R³ is chlorine, n is zero and R⁶ is a phenyl ring, which carries 1 or 2 radicals R¹² as defined in the rows of Table A;

### Table 5

Compounds of the formula IaE, in which R¹, R⁴ and R⁵ are hydrogen, R² is methoxy, R³ is methyl, n is zero and R⁶ is a phenyl ring, which carries 1 or 2 radicals R¹² as defined in the rows of Table A;

### Table 6

Compounds of the formula IaF, in which R¹, R², R⁴ and R⁵ are hydrogen, R³ is methoxy, n is zero and R⁶ is a phenyl ring, which carries 1 or 2 radicals R¹² as defined in the rows of Table A;

### Table 7

Compounds of the formula IaG, in which R¹, R³, R⁴ and R⁵ are hydrogen, R² is methoxy, n is zero and R⁶ is a phenyl ring, which carries 1 or 2 radicals R¹² as defined in the rows of Table A;

### Table 8

Compounds of the formula IaH, in which R¹, R⁴ and R⁵ are hydrogen, R² is methoxy, R³ is methoxy, n is zero and R⁶ is a phenyl ring, which carries 1 or 2 radicals R¹² as defined in the rows of Table A;

### Table 9

Compounds of the formula IaK, in which R¹, R³, R⁴ and R⁵ are hydrogen, R² is methyl, n is zero and R⁶ is a phenyl ring, which carries 1 or 2 radicals R¹² as defined in the rows of Table A;

### Table 10

Compounds of the formula IaL, in which R¹, R², R⁴ and R⁵ are hydrogen, R³ is methyl, n is zero and R⁶ is a phenyl ring, which carries 1 or 2 radicals R¹² as defined in the rows of Table A;

### Table 11

Compounds of the formula IaM, in which R¹, R⁴ and R⁵ are hydrogen, R² is methyl, R³ is methyl, n is zero and R⁶ is a phenyl ring, which carries 1 or 2 radicals R¹² as defined in the rows of Table A;

### Table 12

Compounds of the formula IaN, in which R¹, R², R⁴ and R⁵ are hydrogen, R³ is OCHF₂, n is zero and R⁶ is a phenyl ring, which carries 1 or 2 radicals R¹² as defined in the rows of Table A;

### Table 13

Compounds of the formula IaP, in which R¹, R³, R⁴ and R⁵ are hydrogen, R² is OCHF₂, n is zero and R⁶ is a phenyl ring, which carries 1 or 2 radicals R¹² as defined in the rows of Table A;

### Table 14

Compounds of the formula IbA, in which R¹, R², R³, R⁴ and R⁵ are hydrogen, n is one, R⁷ is methyl and R⁶ is a phenyl ring, which carries 1 or 2 radicals R¹² as defined in the rows of Table A;

### Table 15

Compounds of the formula IbB, in which R¹, R², R⁴ and R⁵ are hydrogen, R³ is chlorine, n is n is one, R⁷ is methyl and R⁶ is a phenyl ring, which carries 1 or 2 radicals R¹² as defined in the rows of Table A;

### Table 16

Compounds of the formula IbC, in which R¹, R³, R⁴ and R⁵ are hydrogen, R² is chlorine, n is one, R⁷ is methyl and R⁶ is a phenyl ring, which carries 1 or 2 radicals R¹² as defined in the rows of Table A;

### Table 17

Compounds of the formula IbD, in which R¹, R⁴ and R⁵ are hydrogen, R² is chlorine, R³ is chlorine, n is one, R⁷ is methyl and R⁶ is a phenyl ring, which carries 1 or 2 radicals R¹² as defined in the rows of Table A;

### Table 18

Compounds of the formula IbE, in which R¹, R⁴ and R⁵ are hydrogen, R² is methoxy, R³ is methyl, n is one, R⁷ is methyl and R⁶ is a phenyl ring, which carries 1 or 2 radicals R¹² as defined in the rows of Table A;

### Table 19

Compounds of the formula IbF, in which R¹, R², R⁴ and R⁵ are hydrogen, R³ is methoxy, n is one, R⁷ is methyl and R⁶ is a phenyl ring, which carries 1 or 2 radicals R¹² as defined in the rows of Table A;

### Table 20

Compounds of the formula IbG, in which R¹, R³, R⁴ and R⁵ are hydrogen, R² is methoxy, n is one, R⁷ is methyl and R⁶ is a phenyl ring, which carries 1 or 2 radicals R¹² as defined in the rows of Table A;

### Table 21

Compounds of the formula IbH, in which R¹, R⁴ and R⁵ are hydrogen, R² is methoxy, R³ is methoxy, n is one, R⁷ is methyl and R⁶ is a phenyl ring, which carries 1 or 2 radicals R¹² as defined in the rows of Table A;

### Table 22

Compounds of the formula IbK, in which R¹, R³, R⁴ and R⁵ are hydrogen, R² is methyl, n is one, R⁷ is methyl and R⁶ is a phenyl ring, which carries 1 or 2 radicals R¹² as defined in the rows of Table A;

### Table 23

Compounds of the formula IbL, in which R¹, R², R⁴ and R⁵ are hydrogen, R³ is methyl, n is one, R⁷ is methyl and R⁶ is a phenyl ring, which carries 1 or 2 radicals R¹² as defined in the rows of Table A;

### Table 24

Compounds of the formula IbM, in which R¹, R⁴ and R⁵ are hydrogen, R² is methyl, R³ is methyl, n is one, R⁷ is methyl and R⁶ is a phenyl ring, which carries 1 or 2 radicals R¹² as defined in the rows of Table A;

### Table 25

Compounds of the formula IbN, in which R¹, R², R⁴ and R⁵ are hydrogen, R³ is OCHF₂, n is one, R⁷ is methyl and R⁶ is a phenyl ring, which carries 1 or 2 radicals R¹² as defined in the rows of Table A;

### Table 26

Compounds of the formula IbP, in which R¹, R³, R⁴ and R⁵ are hydrogen, R² is OCHF₂, n is one, R⁷ is methyl and R⁶ is a phenyl ring, which carries 1 or 2 radicals R¹² as defined in the rows of Table A;

### Table 27

Compounds of the formula IcA, in which R¹, R², R³, R⁴ and R⁵ are hydrogen, n is one, R⁷ is methoxy and R⁶ is a phenyl ring, which carries 1 or 2 radicals R¹² as defined in the rows of Table A;

### Table 28

Compounds of the formula IcB, in which R¹, R², R⁴ and R⁵ are hydrogen, R³ is chlorine, n is one, R⁷ is methoxy and R⁶ is a phenyl ring, which carries 1 or 2 radicals R¹² as defined in the rows of Table A;

### Table 29

Compounds of the formula IcC, in which R¹, R³, R⁴ and R⁵ are hydrogen, R² is chlorine, n is one, R⁷ is methoxy and R⁶ is a phenyl ring, which carries 1 or 2 radicals R¹² as defined in the rows of Table A;

### Table 30

Compounds of the formula IcD, in which R¹, R⁴ and R⁵ are hydrogen, R² is chlorine, R³ is chlorine, n is one, R⁷ is methoxy and R⁶ is a phenyl ring, which carries 1 or 2 radicals R¹² as defined in the rows of Table A;

### Table 31

Compounds of the formula IcE, in which R¹, R⁴ and R⁵ are hydrogen, R² is methoxy, R³ is methyl, n is one, R⁷ is methoxy and R⁶ is a phenyl ring, which carries 1 or 2 radicals R¹² as defined in the rows of Table A;

### Table 32

Compounds of the formula IcF, in which R¹, R², R⁴ and R⁵ are hydrogen, R³ is methoxy, n is one, R⁷ is methoxy and R⁶ is a phenyl ring, which carries 1 or 2 radicals R¹² as defined in the rows of Table A;

### Table 33

Compounds of the formula IcG, in which R¹, R³, R⁴ and R⁵ are hydrogen, R² is methoxy, n is one, R⁷ is methoxy and R⁶ is a phenyl ring, which carries 1 or 2 radicals R¹² as defined in the rows of Table A;

### Table 34

Compounds of the formula IcH, in which R¹, R⁴ and R⁵ are hydrogen, R² is methoxy, R³ is methoxy, n is one, R⁷ is methoxy and R⁶ is a phenyl ring, which carries 1 or 2 radicals R¹² as defined in the rows of Table A;

### Table 35

Compounds of the formula IcK, in which R¹, R³, R⁴ and R⁵ are hydrogen, R² is methyl, n is one, R⁷ is methoxy and R⁶ is a phenyl ring, which carries 1 or 2 radicals R¹² as defined in the rows of Table A;

### Table 36

Compounds of the formula IcL, in which R¹, R², R⁴ and R⁵ are hydrogen, R³ is methyl, n is one, R⁷ is methoxy and R⁶ is a phenyl ring, which carries 1 or 2 radicals R¹² as defined in the rows of Table A;

### Table 37

Compounds of the formula IcM, in which R¹, R⁴ and R⁵ are hydrogen, R² is methyl, R³ is methyl, n is one, R⁷ is methoxy and R⁶ is a phenyl ring, which carries 1 or 2 radicals R¹² as defined in the rows of Table A;

### Table 38

Compounds of the formula IcN, in which R¹, R², R⁴ and R⁵ are hydrogen, R³ is OCHF₂, n is one, R⁷ is methoxy and R⁶ is a phenyl ring, which carries 1 or 2 radicals R¹² as defined in the rows of Table A;

### Table 39

Compounds of the formula IcP, in which R¹, R³, R⁴ and R⁵ are hydrogen, R² is OCHF₂, n is one, R⁷ is methoxy and R⁶ is a phenyl ring, which carries 1 or 2 radicals R¹² as defined in the rows of Table A;

### Table 40

Compounds of the formula IdA, in which R¹, R², R³, R⁴ and R⁵ are hydrogen, n is one, R⁷ is chlorine and R⁶ is a phenyl ring, which carries 1 or 2 radicals R¹² as defined in the rows of Table A;

### Table 41

Compounds of the formula I dB, in which R¹, R², R⁴ and R⁵ are hydrogen, R³ is chlorine, n is one, R⁷ is chlorine and R⁶ is a phenyl ring, which carries 1 or 2 radicals R¹² as defined in the rows of Table A;

### Table 42

Compounds of the formula IdC, in which R¹, R³, R⁴ and R⁵ are hydrogen, R² is chlorine, n is one, R⁷ is chlorine and R⁶ is a phenyl ring, which carries 1 or 2 radicals R¹² as defined in the rows of Table A;

### Table 43

Compounds of the formula IdD, in which R¹, R⁴ and R⁵ are hydrogen, R² is chlorine, R³ is chlorine, n is one, R⁷ is chlorine and R⁶ is a phenyl ring, which carries 1 or 2 radicals R¹² as defined in the rows of Table A;

### Table 44

Compounds of the formula IdE, in which R¹, R⁴ and R⁵ are hydrogen, R² is methoxy, R³ is methyl, n is one, R⁷ is chlorine and R⁶ is a phenyl ring, which carries 1 or 2 radicals R¹² as defined in the rows of Table A;

### Table 45

Compounds of the formula IdF, in which R¹, R², R⁴ and R⁵ are hydrogen, R³ is methoxy, n is one, R⁷ is chlorine and R⁶ is a phenyl ring, which carries 1 or 2 radicals R¹² as defined in the rows of Table A;

### Table 46

Compounds of the formula IdG, in which R¹, R³, R⁴ and R⁵ are hydrogen, R² is methoxy, n is one, R⁷ is chlorine and R⁶ is a phenyl ring, which carries 1 or 2 radicals R¹² as defined in the rows of Table A;

### Table 47

Compounds of the formula IdH, in which R¹, R⁴ and R⁵ are hydrogen, R² is methoxy, R³ is methoxy, n is one, R⁷ is chlorine and R⁶ is a phenyl ring, which carries 1 or 2 radicals R¹² as defined in the rows of Table A;

### Table 48

Compounds of the formula IdK, in which R¹, R³, R⁴ and R⁵ are hydrogen, R² is methyl, n is one, R⁷ is chlorine and R⁶ is a phenyl ring, which carries 1 or 2 radicals R¹² as defined in the rows of Table A;

### Table 49

Compounds of the formula IdL, in which R¹, R², R⁴ and R⁵ are hydrogen, R³ is methyl, n is one, R⁷ is chlorine and R⁶ is a phenyl ring, which carries 1 or 2 radicals R¹² as defined in the rows of Table A;

### Table 50

Compounds of the formula IdM, in which R¹, R⁴ and R⁵ are hydrogen, R² is methyl, R³ is methyl, n is one, R⁷ is chlorine and R⁶ is a phenyl ring, which carries 1 or 2 radicals R¹² as defined in the rows of Table A;

### Table 51

Compounds of the formula IdN, in which R¹, R², R⁴ and R⁵ are hydrogen, R³ is OCHF₂, n is one, R⁷ is chlorine and R⁶ is a phenyl ring, which carries 1 or 2 radicals R¹² as defined in the rows of Table A;

### Table 52

Compounds of the formula IdP, in which R¹, R³, R⁴ and R⁵ are hydrogen, R² is OCHF₂, n is one, R⁷ is chlorine and R⁶ is a phenyl ring, which carries 1 or 2 radicals R¹² as defined in the rows of Table A;

### Table 53

Compounds of the formula IeA, in which R¹, R², R³, R⁴ and R⁵ are hydrogen, n is one, R⁷ is trifluoromethyl and R⁶ is a phenyl ring, which carries 1 or 2 radicals R¹² as defined in the rows of Table A;

### Table 54

Compounds of the formula IeB, in which R¹, R², R⁴ and R⁵ are hydrogen, R³ is chlorine, n is one, R⁷ is trifluoromethyl and R⁶ is a phenyl ring, which carries 1 or 2 radicals R¹² as defined in the rows of Table A;

### Table 55

Compounds of the formula IeC, in which R¹, R³, R⁴ and R⁵ are hydrogen, R² is chlorine, n is one, R⁷ is trifluoromethyl and R⁶ is a phenyl ring, which carries 1 or 2 radicals R¹² as defined in the rows of Table A;

### Table 56

Compounds of the formula IeD, in which R¹, R⁴ and R⁵ are hydrogen, R² is chlorine, R³ is chlorine, n is one, R⁷ is trifluoromethyl and R⁶ is a phenyl ring, which carries 1 or 2 radicals R¹² as defined in the rows of Table A;

### Table 57

Compounds of the formula IeE, in which R¹, R⁴ and R⁵ are hydrogen, R² is methoxy, R³ is methyl, n is one, R⁷ is trifluoromethyl and R⁶ is a phenyl ring, which carries 1 or 2 radicals R¹² as defined in the rows of Table A;

### Table 58

Compounds of the formula IeF, in which R¹, R², R⁴ and R⁵ are hydrogen, R³ is methoxy, n is one, R⁷ is trifluoromethyl and R⁶ is a phenyl ring, which carries 1 or 2 radicals R¹² as defined in the rows of Table A;

### Table 59

Compounds of the formula IeG, in which R¹, R³, R⁴ and R⁵ are hydrogen, R² is methoxy, n is one, R⁷ is trifluoromethyl and R⁶ is a phenyl ring, which carries 1 or 2 radicals R¹² as defined in the rows of Table A;

### Table 60

Compounds of the formula IeH, in which R¹, R⁴ and R⁵ are hydrogen, R² is methoxy, R³ is methoxy, n is one, R⁷ is trifluoromethyl and R⁶ is a phenyl ring, which carries 1 or 2 radicals R¹² as defined in the rows of Table A;

### Table 61

Compounds of the formula IeK, in which R¹, R³, R⁴ and R⁵ are hydrogen, R² is methyl, n is one, R⁷ is trifluoromethyl and R⁶ is a phenyl ring, which carries 1 or 2 radicals R¹² as defined in the rows of Table A;

### Table 62

Compounds of the formula IeL, in which R¹, R², R⁴ and R⁵ are hydrogen, R³ is methyl, n is one, R⁷ is trifluoromethyl and R⁶ is a phenyl ring, which carries 1 or 2 radicals R¹² as defined in the rows of Table A;

### Table 63

Compounds of the formula IeM, in which R¹, R⁴ and R⁵ are hydrogen, R² is methyl, R³ is methyl, n is one, R⁷ is trifluoromethyl and R⁶ is a phenyl ring, which carries 1 or 2 radicals R¹² as defined in the rows of Table A;

### Table 64

Compounds of the formula IeN, in which R¹, R², R⁴ and R⁵ are hydrogen, R³ is OCHF₂, n is one, R⁷ is trifluoromethyl and R⁶ is a phenyl ring, which carries 1 or 2 radicals R¹² as defined in the rows of Table A;

### Table 65

Compounds of the formula IeP, in which R¹, R³, R⁴ and R⁵ are hydrogen, R² is OCHF₂, n is one, R⁷ is trifluoromethyl and R⁶ is a phenyl ring, which carries 1 or 2 radicals R¹² as defined in the rows of Table A;

TABLE A

| No. | R¹² |
|---|---|
| 1 | 2-F |
| 2 | 3-F |
| 3 | 4-F |
| 4 | 2-F, 3-F |
| 5 | 2-F, 4-F |
| 6 | 3-F, 4-F |
| 7 | 2-Cl |
| 8 | 3-Cl |
| 9 | 4-Cl |
| 10 | 2-Cl, 3-Cl |
| 11 | 2-Cl, 4-Cl |
| 12 | 3-Cl, 4-Cl |
| 13 | 2-CH₃ |
| 14 | 3-CH₃ |
| 15 | 4-CH₃ |
| 16 | 2-CH₃, 3-CH₃ |
| 17 | 2-CH₃, 4-CH₃ |
| 18 | 3-CH₃, 4-CH₃ |
| 19 | 2-C₂H₅ |
| 20 | 3-C₂H₅ |
| 21 | 4-C₂H₅ |
| 22 | 2-C₂H₅, 3-C₂H₅ |
| 23 | 2-C₂H₅, 4-C₂H₅ |
| 24 | 3-C₂H₅, 4-C₂H₅ |
| 25 | 2-CH₂CH₂CH₃ |
| 26 | 3-CH₂CH₂CH₃ |
| 27 | 4-CH₂CH₂CH₃ |
| 28 | 2-CH₂CH₂CH₃, 3-CH₂CH₂CH₃ |
| 29 | 2-CH₂CH₂CH₃, 4-CH₂CH₂CH₃ |
| 30 | 3-CH₂CH₂CH₃, 4-CH₂CH₂CH₃ |
| 31 | 2-CH(CH₃)₂ |
| 32 | 3-CH(CH₃)₂ |
| 33 | 4-CH(CH₃)₂ |
| 34 | 2-CH(CH₃)₂, 3-CH(CH₃)₂ |
| 35 | 2-CH(CH₃)₂, 4-CH(CH₃)₂ |
| 36 | 3-CH(CH₃)₂, 4-CH(CH₃)₂ |
| 37 | 4-C(CH₃)₃ |
| 38 | 2-CF₃ |
| 39 | 3-CF₃ |
| 40 | 4-CF₃ |
| 41 | 2-CF₃, 3-CF₃ |
| 42 | 2-CF₃, 4-CF₃ |
| 43 | 3-CF₃, 4-CF₃ |
| 44 | 2-C₂F₅ |
| 45 | 3-C₂F₅ |
| 46 | 4-C₂F₅ |
| 47 | 2-C₂F₅, 3-C₂F₅ |
| 48 | 2-C₂F₅, 4-C₂F₅ |
| 49 | 3-C₂F₅, 4-C₂F₅ |
| 50 | 2-OH |
| 51 | 3-OH |
| 52 | 4-OH |
| 53 | 2-OH, 3-OH |
| 54 | 2-OH, 4-OH |
| 55 | 3-OH, 4-OH |
| 56 | 2-OCH₃ |
| 57 | 3-OCH₃ |
| 58 | 4-OCH₃ |
| 59 | 2-OCH₃, 3-OCH₃ |
| 60 | 2-OCH₃, 4-OCH₃ |
| 61 | 3-OCH₃, 4-OCH₃ |
| 62 | 2-OCF₃ |
| 63 | 3-OCF₃ |
| 64 | 4-OCF₃ |
| 65 | 2-OCF₃, 3-OCF₃ |
| 66 | 2-OCF₃, 4-OCF₃ |
| 67 | 3-OCF₃, 4-OCF₃ |
| 68 | 2-OC₂F₅ |
| 69 | 3-OC₂F₅ |
| 70 | 4-OC₂F₅ |
| 71 | 2-OC₂F₅, 3-OC₂F₅ |
| 72 | 2-OC₂F₅, 4-OC₂F₅ |
| 73 | 3-OC₂F₅, 4-OC₂F₅ |
| 74 | 2-NO₂ |
| 75 | 3-NO₂ |
| 76 | 4-NO₂ |
| 77 | 2-NO₂, 3-NO₂ |
| 78 | 2-NO₂, 4-NO₂ |
| 79 | 3-NO₂, 4-NO₂ |
| 80 | 2-CN |
| 81 | 3-CN |
| 82 | 4-CN |
| 83 | 2-CN, 3-CN |
| 84 | 2-CN, 4-CN |
| 85 | 3-CN, 4-CN |
| 86 | 2-(CO—OCH₃) |
| 87 | 3-(CO—OCH₃) |
| 88 | 4-(CO—OCH₃) |
| 89 | 2-(CO—OC₂H₅) |
| 90 | 3-(CO—OC₂H₅) |
| 91 | 4-(CO—OC₂H₅) |
| 92 | 2-CHO |
| 93 | 3-CHO |
| 94 | 4-CHO |
| 95 | 2-(CO—CH₃) |
| 96 | 3-(CO—CH₃) |
| 97 | 4-(CO—CH₃) |
| 98 | 2-(CO—NH₂) |
| 99 | 3-(CO—NH₂) |
| 100 | 4-(CO—NH₂) |
| 101 | 2-[C(CH₃)═N—OCH₃] |
| 102 | 3-[C(CH₃)═N—OCH₃] |
| 103 | 4-[C(CH₃)═N—OCH₃] |
| 104 | 2-[C(CH₃)═N—OC₂H₅] |
| 105 | 3-[C(CH₃)═N—OC₂H₅] |
| 106 | 4-[C(CH₃)═N—OC₂H₅] |
| 107 | 2-SCH₃ |
| 108 | 3-SCH₃ |
| 109 | 4-SCH₃ |
| 110 | 2-(SO₂—CH₃) |
| 111 | 3-(SO₂—CH₃) |
| 112 | 4-(SO₂—CH₃) |
| 113 | 2-(SO—CH₃) |
| 114 | 3-(SO—CH₃) |
| 115 | 4-(SO—CH₃) |
| 116 | 2-[N(CH₃)₂] |
| 117 | 3-[N(CH₃)₂] |
| 118 | 4-[N(CH₃)₂] |
| 119 | 2-[Si(CH₃)₃] |
| 120 | 3-[Si(CH₃)₃] |
| 121 | 4-[Si(CH₃)₃] |
| 122 | 2-F, 3-Cl |
| 123 | 2-F, 4-Cl |
| 124 | 2-F, 5-Cl |
| 125 | 2-F, 6-Cl |
| 126 | 3-F, 2-Cl |
| 127 | 3-F, 4-Cl |
| 128 | 3-F, 5-Cl |
| 129 | 4-F, 2-Cl |
| 130 | 4-F, 3-Cl |
| 131 | 4-F, 2-CH₃ |
| 132 | 4-Cl, 2-CH₃ |
| 133 | 2-Cl, 4-OCH₃ |
| 134 | 3-Cl, 4-OCH₃ |
| 135 | 2-F, 4-OCH₃ |
| 136 | 3-F, 4-OCH₃ |
| 137 | 3,4 (O—CH₂—O) |

The compounds I according to the invention can be prepared by analogy to the methods described in the art.

Advantageously, they are obtained from pyridine derivatives of the formula II.

A suitable process for the preparation of the compounds I comprises the reaction of compounds II with sulfonic acids or sulfonic acid derivatives of the formula III, under basic conditions as described in the following reaction scheme:

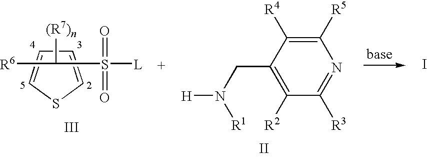

In formulae II and III, n and the radicals R¹, R², R³, R⁴, R⁵, R⁶ and Rare as defined above. In formula III, L is a suitable leaving group such as hydroxyl or halogen, preferably chlorine.

This reaction is usually carried out at temperatures of from (−30)° C. to 120° C., preferably from (−10)° C. to 100° C., in an inert organic solvent in the presence of a base [cf. Lieb. Ann. Chem. 641 (1990)].

Suitable solvents include aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert.-butyl methyl ether, dioxane, anisole and tetrahydrofuran, nitriles, such as acetonitrile and propionitrile, ketones, such as acetone, methyl ethyl ketone, diethyl ketone and tert.-butyl methyl ketone, and also dimethyl sulfoxide, dimethylformamide and dimethylacetamide, particularly preferably diisopropyl ether, diethyl ether and tetrahydrofuran. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines.

Particular preference is given to pyridine, triethylamine and potassium carbonate.

The bases are generally employed in catalytic amounts; however, they can also be employed in equimolar amounts, in excess or, if appropriate, as solvent.

The starting materials are generally reacted with one another in equimolar amounts. In terms of yield, it may be advantageous to use an excess of II, based on III.

Compounds, wherein R⁶ is optionally substituted phenyl may also be prepared from compounds I, wherein R⁶ is halogen, in particular bromine, by a coupling reaction such as a Stille-coupling or under the conditions of a Suzuki-coupling, e.g. by the reaction shown in the following scheme:

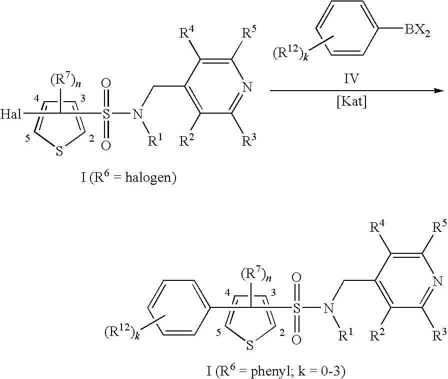

In the scheme, the variables R¹, R², R³, R⁴, R⁵, and R¹² are as defined above. The variable k is 0,1, 2 or 3. Hal is halogen, in particular bromine. X in Formla IV is OH or C₁-C₄-alkoxy. Kat is a transition metal catalyst, in particular a Pd-catalyst. Reaction conditions can be taken from the working examples or from Suzuki et al., Chem. Rev. 95, 2457-2483 (1995) and the literature cited therein.

The coupling reaction is carried out under the same conditions as the condensation of the compounds II and III.

The Suzuki-coupling of a compound I, wherein R⁶ is halogen, with a boronic acid derivatives IV or an ester thereof is usually carried out at temperatures of from 20° C. to 180° C., preferably from 40° C. to 120° C., in an inert organic solvent in the presence of a base and a platinum metal, in particular a palladium catalyst [cf. Synth. Commun. Vol. 11, p. 513 (1981); Acc. Chem. Res. Vol. 15, pp. 178-184 (1982); Chem. Rev. Vol. 95, pp. 2457-2483 (1995); Organic Letters Vol. 6 (16), p. 2808 (2004); WO 2002/42275].

Suitable catalysts are in particular tetrakis(triphenylphosphine)palladium(0); bis(triphenylphosphine)palladium(II) chloride; bis(acetonitrile)palladium(II) chloride; [1,1′-bis(diphenylphosphino)ferrocene]-palladium(II) chloride/methylene chloride (1:1) complex; bis[bis-(1,2-diphenylphosphino)ethane]palladium(0); bis(bis-(1,2-diphenylphosphino)butane]-palladium(II) chloride; palladium(II) acetate; palladium(II) chloride; and palladium(II) acetate/tri-o-tolylphosphine complex; tris-tert.-butyl-phosphine/palladium-dibenzylidene acetone.

Suitable solvents are aliphatic hydrocarbons, such as pentane, hexane, cyclohexane and petroleum ether, aromatic hydrocarbons, such as toluene, o-, m- and p-xylene, ethers, such as diisopropyl ether, tert.-butyl methyl ether, dioxane, anisole and tetrahydrofuran and dimethoxyethane, ketones, such as acetone, methyl ethyl ketone, diethyl ketone and tert.-butyl methyl ketone, and also dimethyl sulfoxide, dimethylformamide and dimethylacetamide, particularly preferably ethers, such as tetrahydrofuran, dioxane and dimethoxyethane. It is also possible to use mixtures of the solvents mentioned.

Suitable bases are, in general, inorganic compounds, such as alkali metal and alkaline earth metal oxides, such as lithium oxide, sodium oxide, calcium oxide and magnesium oxide, alkali metal and alkaline earth metal carbonates, such as lithium carbonate, sodium carbonate, potassium carbonate and calcium carbonate, and also alkali metal bicarbonates, such as sodium bicarbonate, alkali metal and alkaline earth metal alkoxides, such as sodium methoxide, sodium ethoxide, potassium ethoxide and potassium tert.-butoxide, moreover organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines. Particular preference is given to bases such as sodium carbonate, potassium carbonate, cesium carbonate, triethylamine and sodium bicarbonate.

The bases are generally employed in equimolar amounts; however, they can also be employed in excess or, if appropriate, as solvent.

The starting materials are generally reacted with one another in equimolar amounts. In terms of yield, it may be advantageous to use an excess of IV, based on I (R⁶ being halogen).

The intermediate III can be prepared from the respective pyridylhalide V by treatment with alkylmagnesiumhalogenide such as (CH₃)₂CH—MgCl, SO₂ and SO₂Cl₂ as shown in the scheme below.

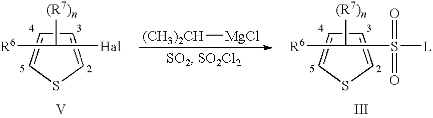

The starting materials required for preparing the compounds I are commercially available or known in the art or they can be prepared by analogy to the methods described in the art.

For example, aminomethylpyridine compounds fo the formula II in which one or more of the radicals R², R³, R⁴ or R⁵ is/are different from hydrogen, such as (halo)alkoxy, (halo)alkylthio, (halo)alkyl, alkenyl, trialkylsilyl or alkynyl, may be prepared starting from halopyridinecarbonitriles by replacing a halogen radical against a radical different from halogen, by conventional nucleophilic substitution reaction or by a coupling reaction, e.g. by treatment with suitable nucleophile such as HNR′ R′ ′, (halo)alkoxide, (halo)alkylthio, a metal organic compound, optionally in the presence of a transition metal catalyst, to obtain the corresponding substituted carbonitrile [cf. Journal of Medicinal Chemistry, 22(11), 1284-90 (1979); U.S. Pat. No. 4,558,134, Synthesis (6), 763-768 (1996) and Heterocycles 41(4), 675-88 (1995)], and subsequent hydrogenation of the C≡N radical to obtain the corresponding aminomethylpyridine compound II, wherein R¹ is hydrogen [cf. Heterocycles 41(4), 675-88 (1995); Recueil des Travaux Chimiques des Pays-Bas et de la Belgique 52, 55-60 (1933); Acta Poloniae Pharmaceutica 32(3), 265-8 (1975); Journal of Medicinal Chemistry 24(1), 115-17 (1981), P 49173, Heterocycles 41(4), 675-88 (1995), Angewandte Chemie, International Edition, 43(37), 4902-4906 (2004); Journal of Heterocyclic Chemistry 19(6), 1551-2 (1982)]. The subsequent alkylation of the amino methyl nitrogen yields compounds, wherein R¹ is different from hydrogen.

The reaction mixtures are worked up in the customary manner, for example by mixing with water, separating the phases and, if appropriate, chromatographic purification of the crude products. Some of the intermediates and end products are obtained in the form of colorless or slightly brownish viscous oils, which can be purified or freed from volatile components under reduced pressure and at moderately elevated temperature. If the intermediates and end products are obtained as solids, purification can also be carried out by recrystallization or digestion.

The N-oxides may be prepared from the compounds I according to conventional oxidation methods, for example by treating the free thiophene-sulfonic acid picolyl amides I with an organic peracid such as metachloroperbenzoic acid [Journal of Medicinal Chemistry 38(11), 1892-903 (1995), WO 03/64572]; or with inorganic oxidizing agents such as hydrogen peroxide [cf. Journal of Heterocyclic Chemistry 18(7), 1305-8 (1981)] or oxone [cf. Journal of the American Chemical Society 123(25), 5962-5973 (2001)]. The oxidation may lead to pure mono-N-oxides or to a mixture of different N-oxides, which can be separated by conventional methods such as chromatography. Preferably one or two of the pyridine nitrogens in compounds I are oxidized to the corresponding mono- or bis-N-oxides.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus or pest to be controlled.

The compounds I are suitable as fungicides. They are distinguished by an outstanding effectiveness against a broad spectrum of phytopathogenic fungi, especially from the classes of the Ascomycetes, Basidiomycetes, Deuteromycetes and

Peronosporomycetes (syn. Oomycetes) and Fungi imperfecti. Some are systemically effective and they can be used in crop protection as foliar fungicides, fungicides for seed dressing and soil fungicides.

They are particularly important in the control of a multitude of fungi on various cultivated plants, such as wheat, rye, barley, triticale, oats, rice, corn, grass, bananas, cotton, soybeans, coffee, sugar cane, grapevines, fruit and ornamental plants, and vegetables, such as cucumbers, beans, tomatoes, potatoes and cucurbits, and on the seeds of these plants. They can also be used in crops which are tolerant against attack by insects or fungi or herbicide applications due to breeding, including genetic engineering methods. Moreover, they are suitable for controlling Botryosphaeria species, Cylindrocarpon species, Eutypa lata, Neonectria liriodendri and Stereum hirsutum, which attack, inter alia, wood or the roots of grapevines.

The compounds I are suitable for controlling Alternaria species on vegetables, rapeseed, sugarbeet, fruit, rice, soybeans and on potatoes (for example, A. solani or A. alternata) and tomatoes (for example, A. solani or A. alternata) and Alternaria ssp. (ear black) on wheat.

The compounds I are suitable for controlling Aphanomyces species on sugarbeet and vegetables.

The compounds I are suitable for controlling Ascochyta species on cereals and vegetables, for example Ascochyta tritici (leaf spot) on wheat.

The compounds I are suitable for controlling Bipolaris and Drechslera species on corn (for example, D. Maydis), cereals, rice and lawns.

The compounds I are suitable for controlling Blumeria graminis (powdery mildew) on cereals (for example, wheat or barley).

The compounds I are suitable for controlling Botrytis cinerea (gray mold) on strawberries, vegetables, flowers, grapevines and wheat (ear mildew).

The compounds I are suitable for controlling Bremia lactucae on lettuce.

The compounds I are suitable for controlling Cercospora species on corn, rice, sugarbeet and, for example, Cercospora sojina (leaf spot) or Cercospora kikuchii (leaf spot) on soybeans.

The compounds I are suitable for controlling Cladosporium herbarum (ear black) in wheat.

The compounds I are suitable for controlling Cochliobolus species on corn, cereals (for example, Cochliobolus sativus) and rice (for example, Cochliobolus miyabeanus).

The compounds I are suitable for controlling Colletotricum species on cotton and, for example, Colletotrichum truncatum (Antracnose) on soybeans.

The compounds I are suitable for controlling corynespora cassiicola (leaf spot) on soybeans.

The compounds I are suitable for controlling Dematophora necatrix (root/stem rot) on soybeans.

The compounds I are suitable for controlling Diaporthe phaseolorum (stem disease) on soybeans.

The compounds I are suitable for controlling Drechslera species, Pyrenophora species on corn, cereals, rice and lawns, on barley (for example, D. teres) and on wheat (for example, D. tritici-repentis).

The compounds I are suitable for controlling Esca on grapevines, caused by Phaeoacremonium chlamydosporium, Ph. Aleophilum, and Formitipora punctata (syn. Phellinus punctatus).

The compounds I are suitable for controlling Elsinoe ampelina on grapevines.

The compounds I are suitable for controlling Epicoccum spp. (ear black) on wheat.

The compounds I are suitable for controlling Exserohilum species on corn.

The compounds I are suitable for controlling Erysiphe cichoracearum and Sphaerotheca fuliginea on cucumbers.

The compounds I are suitable for controlling Fusarium and Verticillium species on various plants: for example, F. graminearum or F. culmorum (root rot) on cereals (for example, wheat or barley) or, for example, F. oxysporum tomatoes and Fusarium solani (stem disease) on soybeans.

The compounds I are suitable for controlling Gaeumanomyces graminis (root black) on cereals (for example, wheat or barley).

The compounds I are suitable for controlling Gibberella species on cereals and rice (for example Gibberella fujikuroi).

The compounds I are suitable for controlling Glomerella cingulata on grapevines and other plants.

The compounds I are suitable for controlling Grainstaining complex on rice.

The compounds I are suitable for controlling Guignardia budwelli on grapevines.

The compounds I are suitable for controlling Helminthosporium species on corn and rice.

The compounds I are suitable for controlling Isariopsis clavispora on grapevines.

The compounds I are suitable for controlling Macrophomina phaseolina (root/stem rot) on soybeans.

The compounds I are suitable for controlling Michrodochium nivale (snow mold) on cereals (for example, wheat or barley).

The compounds I are suitable for controlling Microsphaera diffusa (powdery mildew) on soybeans.

The compounds I are suitable for controlling Mycosphaerella species on cereals, bananas and peanuts, such as, for example, M. graminicola on wheat or M. fijiensis on bananas.

The compounds I are suitable for controlling Peronospora species on cabbage (for example, P. brassicae), bulbous plants (for example, P. destructor) and, for example, Peronospora manshurica (downy mildew) on soybeans.

The compounds I are suitable for controlling Phakopsara pachyrhizi (soya rust) and Phakopsara meibomiae (soya rust) on soybeans.

The compounds I are suitable for controlling Phialophora gregata (stem disease) on soybeans.

The compounds I are suitable for controlling Phomopsis species on sunflowers, grapevines (for example, P. viticola) and soybeans (for example, Phomopsis phaseoli).

The compounds I are suitable for controlling Phytophthora species on various plants, for example, P. capsici on bell peppers, Phytophthora megasperma (leaf/stem rot) on soybeans, Phytophthora infestans on potatoes and tomatoes.

The compounds I are suitable for controlling Plasmopara viticola on grapevines.

The compounds I are suitable for controlling Podosphaera leucotricha on apples.

The compounds I are suitable for controlling Pseudocercosporella herpotrichoides (eyespot) on cereals (wheat or barley).

The compounds I are suitable for controlling Pseudoperonospora on various plants, for example, P. cubensis on cucumbers or P. humili on hops.

The compounds I are suitable for controlling Pseudopezicula tracheiphilai on grapevines.

The compounds I are suitable for controlling Puccinia species on various plants, for example, P. triticina, P. striformins, P. hordei or P. graminis on cereals (for example, wheat or barley), or on asparagus (for example, P. asparagi).

The compounds I are suitable for controlling Pyricularia oryzae, Corticium sasakii, Sarocladium oryzae, S. attenuatum, Pyrenophora tritici-repentis (leaf spot) on wheat or Pyrenophora teres (net blotch) on barley.

The compounds I are suitable for controlling Entyloma oryzae on rice.

The compounds I are suitable for controlling Pyricularia grisea on lawns and cereals.

The compounds I are suitable for controlling Pythium spp. on lawns, rice, corn, wheat, cotton, rapeseed, sunflowers, sugarbeet, vegetables and other plants (for example, P. ultiumum or P. aphanidermatum).

The compounds I are suitable for controlling Ramularia collo-cygni (Ramularia/sunburn complex/physiological leaf spots) on barley.

The compounds I are suitable for controlling Rhizoctonia species on cotton, rice, potatoes, lawns, corn, rapeseed, potatoes, sugarbeet, vegetables and on various plants for example, Rhizoctonia solani (root/stem rot) on soybeans or Rhizoctonia cerealis (sharp eyspot) on wheat or barley.

The compounds I are suitable for controlling Rhynchosporium secalis on barley (leaf spot), rye and triticale.

The compounds I are suitable for controlling Sclerotinia species on rapeseed and sunflowers, and, for example, Sclerotinia sclerotiorum (stem disease) or Sclerotinia rolfsii (stem disease) on soybeans.

The compounds I are suitable for controlling Septoria glycines (leaf spot) on soybeans.

The compounds I are suitable for controlling Septoria tritici (leaf septoria) and Stagonospora nodorum on wheat.

The compounds I are suitable for controlling Erysiphe (syn. Uncinula) necator on grapevines.

The compounds I are suitable for controlling Setospaeria species on corn and lawns.

The compounds I are suitable for controlling Sphacelotheca reilinia on corn.

The compounds I are suitable for controlling Stagonospora nodorum (ear septoria) on wheat.

The compounds I are suitable for controlling Thievaliopsis species on soybeans and cotton

The compounds I are suitable for controlling Tilletia species on cereals.

The compounds I are suitable for controlling Typhula incarnata (snow rot) on wheat or barley.

The compounds I are suitable for controlling Ustilago species on cereals, corn (for example, U. maydis) and sugarcane.

The compounds I are suitable for controlling Venturia species (scab) on apples (for example, V. inaequalis) and pears.

The compounds I are also suitable for controlling harmful fungi in the protection of materials (e.g. wood, paper, paint dispersions, fiber or fabrics) and in the protection of stored products. As to the protection of wood, the following harmful fungi are worthy of note: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of stored products the following yeast fungi are worthy of note: Candida spp. and Saccharomyces cerevisae.

The compounds I, their N-oxides or their salts are employed by treating the fungi or the plants, seeds, materials or the soil to be protected from fungal attack with a fungicidally effective amount of the active compounds. The application can be carried out both before and after the infection of the materials, plants, seeds or soil by the fungi.

The fungicidal compositions generally comprise between 0.1 and 95%, preferably between 0.5 and 90%, by weight of active compound. The active compounds are employed in a purity of from 90% to 100%, preferably from 95% to 100% (according to NMR spectrum).

When employed in plant protection, the amounts applied are, depending on the kind of effect desired, between 0.01 and 2.0 kg of active compound per ha.

In seed treatment, for example by dusting, coating or drenching seed, amounts of active compound of from 1 to 1000 g, preferably from 5 to 100 g, per 100 kilogram of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

In addition the compounds of the formula I may also be used in cultures which can tolerate insecticidal or fungal attack due to cultivation, including of genetic engineering.

The compounds of the formula I are furthermore suitable for controlling pests from the classes of the insects, arachnids and nematodes effectively. They can be used as pesticides in crop protection and in the sectors of hygiene and the protection of stored products and the veterinary sector.

They may act by contact or may be stomach-acting, or have systemic or residual action. Contact action means that the pest is killed by coming into contact with a compound I or with material that releases compound I. Stomach-acting means that the pest is killed if it ingests a pesticidally effective amount of the compound I or material containing a pesticidally effective amount of compound I. Systemic action means that the compound is absorbed into the plant tissues of treated plant and the pest is controlled, if it eats plant tissue or sucks plant-sap.

Compounds I are in particular suitable for controlling the following insect pests:

insects from the order of Lepidoptera, for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera eridania, Spodoptera frugiperda, Spodoptera littoralis, Spodoptera litura, Thaumatopoea pityocampa, Tortrix viridana, Trichoplusia ni and Zeiraphera canadensis,

from the order of Coleoptera (beetles), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria,

from the order of Diptera, for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa,

from the order of Thysanoptera (thrips), e.g. Dichromothrips spp., Frankliniella fusca, Frankliniella occidentalis, Frankliniella tritici, Scirtothrips citri, Thrips oryzae, Thrips palmi and Thrips tabaci,

ants, bees, wasps, sawflies from the order of (Hymenoptera) e.g. Athalia rosae, Atta cephalotes, Atta cephalotes, Atta laevigata, Atta robusta, Atta capiguara, Atta sexdens, Atta texana, Crematogaster spp., Hoplocampa minuta, Hoplocampa testudinea, Monomorium pharaonis, Solenopsis geminata, and Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,

from the order of Homoptera, e.g. Acyrthosiphon onobrychis, Adelges laricis, Aphidula nasturtii, Aphis craccivora, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisa tabaci, Bemisa argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla pin, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii,

from the order of Isoptera (termites), e.g. Calotermes flavicollis, Heterotermes aureus, Leucotermes flavipes, Reticulitermes flavipes, Reticulitermes virginicus, Reticulitermes lucifugus, aund Termes natalensis, and Coptotermes formosanus,

cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla pin, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus,

crickets, grasshoppers, locusts from the order of (Orthoptera), e.g. Acheta domestica, Blatta orientalis, Blattella germanica, Calliptamus italicus, Chortoicetes terminifera, Dociostaurus maroccanus, Forficula auricularia, Gryllotalpa gryllotalpa, Hieroglyphus daganensis, Kraussaria angulifera, Locusta migratoria, Locustana pardalina, Melanoplus bivittatus, Melanoplus femur-rubrum, Melanoplus mexicanus, Melanoplus sanguinipes, Melanoplus spretus, Nomadacris septemfasciata, Oedaleus senegalensis, Periplaneta americana, Schistocerca americana, Schistocerca peregrina, Schistocerca gregaria, Stauronotus maroccanus and, Tachycines asynamorus, Tachycines asynamorus, Zonozerus variegatus.

The compounds of the formula I and their salts are also useful for controlling arachnids (Arachnoidea), such as acarians (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus hermsi, Ornithodorus turicata, Ornithonyssus bacoti, Ornithodorus moubata, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis.and Oligonychus pratensis; Araneida, e.g. Latrodectus mactans, and Loxosceles reclusa,

fleas (Siphonaptera), e.g. Ctenocephalides felis, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Scutigera coleoptrata,

millipedes (Diplopoda), e.g. Narceus spp.,

earwigs (Dermaptera), e.g. forficula auricularia,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

The compounds of the formula I, their N-oxides and their salts are also useful for controlling nematodes, for example, root gall nematodes, e.g. Meloidogyne hapla, Meloidogyne incognita, Meloidogyne javanica, cyst-forming nematodes, e.g. Globodera rostochiensis, Heterodera avenae, Heterodera glycines, Heterodera schachtii, Heterodera trifolii, stem and leaf nematodes, e.g. Belonolaimus longicaudatus, Ditylenchus destructor, Ditylenchus dipsaci, Heliocotylenchus multicinctus, Longidorus elongatus, Radopholus similis, Rotylenchus robustus, Trichodorus primitivus, Tylenchorhynchus claytoni, Tylenchorhynchus dubius, Pratylenchus neglectus, Pratylenchus penetrans, Pratylenchus curvitatus and Pratylenchus goodeyi.

Compounds of the formula I are particularly useful for controlling insects of the order Lepidoptera.

In general, the insecticidal compositions comprise from 0.01 to 95% by weight, preferably from 0.1 to 90% by weight, of the active compound. The active compounds are employed in a purity of from 90% to 100%, preferably from 95% to 100% (according to NMR spectrum).

Under outdoor conditions, the active compound application rate for controlling pests is from 0.1 to 2.0, preferably from 0.2 to 1.0, kg/ha.

The compounds I, their N-oxides and salts can be converted into customary formulations (agricultural formulations), e.g. solutions, emulsions, suspensions, dusts, powders, pastes and granules. The application form depends on the particular intended purpose; in each case, it should ensure a fine and uniform distribution of the compound according to the invention.

The formulations are prepared in a known manner, e.g. by extending the active ingredient with solvents and/or carriers, if desired using emulsifiers and dispersants. Solvents/auxiliaries, which are suitable, are essentially:
water, aromatic solvents (for example Solvesso® products, xylene), paraffins (for example mineral fractions), alcohols (for example methanol, butanol, pentanol, benzyl alcohol), ketones (for example cyclohexanone, gamma-butyrolactone), pyrrolidones (N-methylpyrrolidone, N-octylpyrrolidone), acetates (glycol diacetate), glycols, fatty acid dimethylamides, fatty acids and fatty acid esters. In principle, solvent mixtures may also be used.
carriers such as ground natural minerals (e.g. kaolins, clays, talc, chalk) and ground synthetic minerals (e.g. highly disperse silica, silicates); emulsifiers such as nonionic and anionic emulsifiers (e.g. polyoxyethylene fatty alcohol ethers, alkyl-sulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Also anti-freezing agents such as glycerin, ethylene glycol and propylene glycol can be added to the formulation.

Suitable antifoaming agents are, for example, those based on silicone or magnesium stearate.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

Formulations for the treatment of seed may additionally comprise binders and/or gelling agents and, if appropriate, colorants.

Binders may be added to increase the adhesion of the active compounds on the seed after the treatment. Suitable binders are, for example, EO/PO block copolymer surfactants, but also polyvinyl alcohols, polyvinylpyrrolidones, polyacrylates, polymethacrylates, polybutenes, polyisobutylenes, polystyrenes, polyethylenamines, polyethylenamides, polyethylenimines (Lupasol®, Polymin®), polyethers, polyurethanes, polyvinyl acetates, tylose and copolymers of these polymers.

A suitable gelling agent is, for example, carrageen (Satiagel®).

In general, the formulations comprise from 0.01 to 95% by weight, preferably from 0.1 to 90% by weight, of the active compound. The active compounds are employed in a purity of from 90% to 100%, preferably from 95% to 100% (according to NMR spectrum).

The concentrations of active compound in the ready-for-use preparations can be varied within relatively wide ranges. In general, they are between 0.0001 and 10%, preferably between 0.01 and 1%.

The active compounds can also be used with great success in the ultra-low volume (ULV) process, it being possible to apply formulations with more than 95% by weight of active compound or even the active compound without additives.

For the treatment of seed, the formulations in question give, after two-to-tenfold dilution, active compound concentrations of from 0.01 to 60% by weight, preferably from 0.1 to 40% by weight, in the ready-to-use preparations.

The following are examples of formulations: 1. Products for dilution with water

A) Water-soluble concentrates (SL, LS)

10 parts by weight of a compound I according to the invention are dissolved in 90 parts by weight of water or in a water-soluble solvent. As an alternative, wetting agents or other auxiliaries are added. The active compound dissolves upon dilution with water. In this way, a formulation having a content of 10% by weight of active compound is obtained.

B) Dispersible concentrates (DC)

20 parts by weight of a compound I according to the invention are dissolved in 70 parts by weight of cyclohexanone with addition of 10 parts by weight of a dispersant, for example polyvinylpyrrolidone. Dilution with water gives a dispersion. The active compound content is 20% by weight.

C) Emulsifiable concentrates (EC)

15 parts by weight of a compound I according to the invention are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

D) Emulsions (EW, EO, ES)

25 parts by weight of a compound I according to the invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (Ultraturrax) and made into a homogeneous emulsion. Dilution with water gives an emulsion. The formulation has an active compound content of 25% by weight.

E) Suspensions (SC, OD, FS)

In an agitated ball mill, 20 parts by weight of a compound I according to the invention are comminuted with addition of 10 parts by weight of dispersants and wetting agents and 70 parts by weight of water or an organic solvent to give a fine active compound suspension. Dilution with water gives a stable suspension of the active compound. The active compound content in the formulation is 20% by weight.

F) Water-dispersible granules and water-soluble granules (WG, SG)

50 parts by weight of a compound I according to the invention are ground finely with addition of 50 parts by weight of dispersants and wetting agents and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

G) Water-dispersible powders and water-soluble powders (WP, SP, SS, WS)

75 parts by weight of a compound I according to the invention are ground in a rotor-stator mill with addition of 25 parts by weight of dispersants, wetting agents and silica gel. Dilution with water gives a stable dispersion or solution of the active compound. The active compound content of the formulation is 75% by weight.

H) Gel (GF)

In an agitated ball mill, 20 parts by weight of a compound I according to the invention are comminuted with addition of 10 parts by weight of dispersants, 1 part by weight of a gelling agent wetters and 70 parts by weight of water or of an organic solvent to give a fine suspension of the active compound. Dilution with water gives a stable suspension of the active compound, whereby a formulation with 20% (w/w) of active compound is obtained.

2. Products to be applied undiluted

J) Dustable powders (DP, DS)

5 parts by weight of a compound I according to the invention are ground finely and mixed intimately with 95 parts by weight of finely divided kaolin. This gives a dustable product having an active compound content of 5% by weight.

K) Granules (GR, FG, GG, MG)

0.5 part by weight of a compound I according to the invention is ground finely and associated with 99.5 parts by weight of carriers. Current methods are extrusion, spray-drying or the fluidized bed. This gives granules to be applied undiluted having an active compound content of 0.5% by weight.

L) ULV solutions (UL)

10 parts by weight of a compound I according to the invention are dissolved in 90 parts by weight of an organic solvent, for example xylene. This gives a product to be applied undiluted having an active compound content of 10% by weight.

Water-soluble concentrates (LS), suspensions (FS), dusts (DS), water-dispersible and water-soluble powders (WS, SS), emulsions (ES), emulsifiable concentrates (EC) and gel formulations (GF) are usually used for the treatment of seed. These formulations can be applied to the seed in undiluted or, preferably, diluted form. The application can be carried out before sowing.

The active ingredients can be used as such, in the form of their formulations or the use forms prepared therefrom, eg. in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; it is intended to ensure in each case the finest possible distribution of the active ingredients according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.001 to 1%.

The active ingredients may also be used successfully in the ultra-low-volume process (ULV), it being possible to apply formulations comprising over 95% by weight of active ingredient, or even to apply the active ingredient without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the use form as fungicides with one or more other fungicides results in many cases in an expansion of the fungicidal spectrum of activity or avoidance of resistance development being obtained.

In many cases, synergistic effects are obtained.

The present invention furthermore provides a combination of at least one compound according to the invention and/or an agriculturally acceptable salt thereof and at least one further fungicidal, insecticidal, herbicidal and/or growth-regulating active compound.

The following list of fungicides with which the compounds according to the invention can be applied together is meant to illustrate the possible combinations, but not to limit them:

strobilurins
azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate; carboxamides
carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-3-(4-chlorophenyl)prop-2-ynyloxy]-3-methoxyphenyl)ethyl)-2-ethanesulfonylamino-3-methylbutyramide;
   azoles
triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole; nitrogenous heterocyclyl compounds
pyridines: fluazinam, pyrifenox, 345-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 6-(3,4-dichlorophenyl)-5-methyl-[1,2,4]triazolo[1,5-a]pyrimidin-7-ylamine, 6-(4-tert-butylphenyl)-5-methyl-[1,2,4]triazolo[1,5-a]pyrimidin-7-ylamine, 5-methyl-6-(3,5,5-trimethylhexyl)-[1,2,4]triazolo[1,5-a]pyrimidin-7-ylamine, 5-methyl-6-octyl-[1,2,4]triazolo[1,5-a]pyrimidin-7-ylamine, 5-ethyl-6-octyl-[1,2,4]triazolo[1,5-a]pyrimidin-2,7-diamine, 6-ethyl-5-octyl-[1,2,4]triazolo[1,5-a]pyrimidin-7-ylamine, 5-ethyl-6-octyl-[1,2,4]triazolo[1,5-a]pyrimidin-7-ylamine, 5-ethyl-6-(3,5,5-trimethylhexyl)-[1,2,4]triazolo[1,5-a]pyrimidin-7-ylamine, 6-octyl-5-propyl-[1,2,4]triazolo[1,5-a]pyrimidin-7-ylamine, 5-methoxymethyl-6-octyl-[1,2,4]triazolo[1,5-a]pyrimidin-7-ylamine, 6-octyl-5-trifluoromethyl-[1,2,4]triazolo[1,5-a]pyrimidin-7-ylamine, 5-trifluoromethyl-6-(3,5,5-trimethylhexyl)-[1,2,4]triazolo[1,5-a]pyrimidin-7-ylamine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;
   carbamates and dithiocarbamates
dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;
   other fungicides
guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

Accordingly, the present invention furthermore relates to the compositions listed in Table B, where a row of Table B corresponds in each case to a fungicidal composition comprising a compound of the formula I (component 1), which is preferably one of the compounds described herein as being preferred, and the respective further active compound (component 2) stated in the row in question.

TABLE B

| Row | Component 1 | Component 2 |
|---|---|---|
| B-1 | a compound of the formula I | azoxystrobin |
| B-2 | a compound of the formula I | dimoxystrobin |
| B-3 | a compound of the formula I | enestroburin |
| B-4 | a compound of the formula I | fluoxastrobin |
| B-5 | a compound of the formula I | kresoxim-methyl |
| B-6 | a compound of the formula I | metominostrobin |
| B-7 | a compound of the formula I | picoxystrobin |
| B-8 | a compound of the formula I | pyraclostrobin |
| B-9 | a compound of the formula I | trifloxystrobin |
| B-10 | a compound of the formula I | orysastrobin |
| B-11 | a compound of the formula I | methyl (2-chloro-5-[1-(3-methylbenzyloxy- |
| | | imino)ethyl]benzyl)carbamate |
| B-12 | a compound of the formula I | methyl (2-chloro-5-[1-(6-methylpyridin-2-yl- |
| | | methoxyimino)ethyl]benzyl)carbamate |
| B-13 | a compound of the formula I | methyl 2-(ortho-(2,5-dimethylphenyloxy- |
| | | methylene)phenyl)-3-methoxyacrylate |
| B-14 | a compound of the formula I | benalaxyl |
| B-15 | a compound of the formula I | benodanil |
| B-16 | a compound of the formula I | boscalid |
| B-17 | a compound of the formula I | carboxin |
| B-18 | a compound of the formula I | mepronil |
| B-19 | a compound of the formula I | fenfuram |
| B-20 | a compound of the formula I | fenhexamid |
| B-21 | a compound of the formula I | flutolanil |
| B-22 | a compound of the formula I | furametpyr |
| B-23 | a compound of the formula I | metalaxyl |
| B-24 | a compound of the formula I | ofurace |
| B-25 | a compound of the formula I | oxadixyl |
| B-26 | a compound of the formula I | oxycarboxin |
| B-27 | a compound of the formula I | penthiopyrad |
| B-28 | a compound of the formula I | thifluzamide |
| B-29 | a compound of the formula I | tiadinil |
| B-30 | a compound of the formula I | N-(4′-bromobiphenyl-2-yl)-4-difluoromethyl-2- |
| | | methylthiazole-5-carboxamide |
| B-31 | a compound of the formula I | N-(4′-trifluoromethylbiphenyl-2-yl)-4-di- |
| | | fluoromethyl-2-methylthiazole-5-carboxamide |
| B-32 | a compound of the formula I | N-(4′-chloro-3′-fluorobiphenyl-2-yl)-4-di- |
| | | fluoromethyl-2-methylthiazole-5-carboxamide |
| B-33 | a compound of the formula I | N-(3′,4′-dichloro-4-fluorobiphenyl-2-yl)-3-di- |
| | | fluoromethyl-1-methylpyrazole-4-carboxamide |
| B-34 | a compound of the formula I | N-(3′,4′-dichloro-5-fluorobiphenyl-2-yl)-3-di- |
| | | fluoromethyl-1-methylpyrazole-4-carboxamide |
| B-35 | a compound of the formula I | N-(2-cyanophenyl)-3,4-dichloroisothiazole-5- |
| | | carboxamide |
| B-36 | a compound of the formula I | dimethomorph |
| B-37 | a compound of the formula I | flumorph |
| B-38 | a compound of the formula I | flumetover |
| B-39 | a compound of the formula I | fluopicolide (picobenzamid) |
| B-40 | a compound of the formula I | zoxamide |
| B-41 | a compound of the formula I | carpropamid |
| B-42 | a compound of the formula I | diclocymet |
| B-43 | a compound of the formula I | mandipropamid |
| B-44 | a compound of the formula I | N-(2-(4-[3-(4-chlorophenyl)prop-2-ynyloxy]-3- |
| | | methoxyphenyl)ethyl)-2-methanesulfonyl- |
| | | amino-3-methylbutyramide |
| B-45 | a compound of the formula I | N-(2-(4-[3-(4-Chlorophenyl)prop-2-ynyloxy]-3- |
| | | methoxyphenyl)ethyl)-2-ethanesulfonylamino- |
| | | 3-methylbutyramide |
| B-46 | a compound of the formula I | bitertanol |
| B-47 | a compound of the formula I | bromuconazole |
| B-48 | a compound of the formula I | cyproconazole |
| B-49 | a compound of the formula I | difenoconazole |
| B-50 | a compound of the formula I | diniconazole |
| B-51 | a compound of the formula I | enilconazole |
| B-52 | a compound of the formula I | epoxiconazole |
| B-53 | a compound of the formula I | fenbuconazole |
| B-54 | a compound of the formula I | flusilazole |
| B-55 | a compound of the formula I | fluquinconazole |
| B-56 | a compound of the formula I | flutriafol |
| B-57 | a compound of the formula I | hexaconazol |
| B-58 | a compound of the formula I | imibenconazole |
| B-59 | a compound of the formula I | ipconazole |
| B-60 | a compound of the formula I | metconazol |
| B-61 | a compound of the formula I | myclobutanil |
| B-62 | a compound of the formula I | penconazole |
| B-63 | a compound of the formula I | propiconazole |
| B-64 | a compound of the formula I | prothioconazole |
| B-65 | a compound of the formula I | simeconazole |
| B-66 | a compound of the formula I | tebuconazole |
| B-67 | a compound of the formula I | tetraconazole |
| B-68 | a compound of the formula I | triadimenol |
| B-69 | a compound of the formula I | triadimefon |
| B-70 | a compound of the formula I | triticonazole |
| B-71 | a compound of the formula I | cyazofamid |
| B-72 | a compound of the formula I | imazalil |
| B-73 | a compound of the formula I | pefurazoate |
| B-74 | a compound of the formula I | prochloraz |
| B-75 | a compound of the formula I | triflumizole |
| B-76 | a compound of the formula I | benomyl |
| B-77 | a compound of the formula I | carbendazim |
| B-78 | a compound of the formula I | fuberidazole |
| B-79 | a compound of the formula I | thiabendazole |
| B-80 | a compound of the formula I | ethaboxam |
| B-81 | a compound of the formula I | etridiazole |
| B-82 | a compound of the formula I | hymexazole |
| B-83 | a compound of the formula I | fluazinam |
| B-84 | a compound of the formula I | pyrifenox |
| B-85 | a compound of the formula I | 3-[5-(4-chlorophenyl)-2,3-dimethylisoxazo- |
| | | lidin-3-yl]pyridine |
| B-86 | a compound of the formula I | bupirimate |
| B-87 | a compound of the formula I | cyprodinil |
| B-88 | a compound of the formula I | ferimzone |
| B-89 | a compound of the formula I | fenarimol |
| B-90 | a compound of the formula I | mepanipyrim |
| B-91 | a compound of the formula I | nuarimol |
| B-92 | a compound of the formula I | pyrimethanil |
| B-93 | a compound of the formula I | triforine |
| B-94 | a compound of the formula I | fludioxonil |
| B-95 | a compound of the formula I | fenpiclonil |
| B-96 | a compound of the formula I | aldimorph |
| B-97 | a compound of the formula I | dodemorph |
| B-98 | a compound of the formula I | fenpropimorph |
| B-99 | a compound of the formula I | tridemorph |
| B-100 | a compound of the formula I | iprodione |
| B-101 | a compound of the formula I | procymidone |
| B-102 | a compound of the formula I | vinclozolin |
| B-103 | a compound of the formula I | acibenzolar-S-methyl |
| B-104 | a compound of the formula I | anilazin |
| B-105 | a compound of the formula I | captan |
| B-106 | a compound of the formula I | captafol |
| B-107 | a compound of the formula I | dazomet |
| B-108 | a compound of the formula I | diclomezine |
| B-109 | a compound of the formula I | fenoxanil |
| B-110 | a compound of the formula I | folpet |
| B-111 | a compound of the formula I | fenpropidin |
| B-112 | a compound of the formula I | famoxadone |
| B-113 | a compound of the formula I | fenamidone |
| B-114 | a compound of the formula I | octhilinone |
| B-115 | a compound of the formula I | probenazole |
| B-116 | a compound of the formula I | proquinazid |
| B-117 | a compound of the formula I | pyroquilon |
| B-118 | a compound of the formula I | quinoxyfen |
| B-119 | a compound of the formula I | tricyclazole |
| B-120 | a compound of the formula I | 5-chloro-7-(4-methylpiperidin-1-yl)-6-(2,4,6- |
| | | trifluorophenyl)-[1,2,4]triazolo[1,5-a]pyrimidine |
| B-121 | a compound of the formula I | 6-(3,4-dichlorophenyl)-5-methyl- |
| | | [1,2,4]triazolo[1,5-a]pyrimidin-7-ylamine |
| B-122 | a compound of the formula I | 6-(4-tert-butylphenyl)-5-methyl- |
| | | [1,2,4]triazolo[1,5-a]pyrimidin-7-ylamine |
| B-123 | a compound of the formula I | 5-methyl-6-(3,5,5-trimethylhexyl)- |
| | | [1,2,4]triazolo[1,5-a]pyrimidin-7-ylamine |
| B-124 | a compound of the formula I | 5-methyl-6-octyl-[1,2,4]triazolo[1,5-a]- |
| | | pyrimidin-7-ylamine |
| B-125 | a compound of the formula I | 5-ethyl-6-octyl-[1,2,4]triazolo[1,5-a]pyrimidine- |
| | | 2,7-diamine |
| B-126 | a compound of the formula I | 6-ethyl-5-octyl-[1,2,4]triazolo[1,5-a]pyrimidin- |
| | | 7-ylamine |
| B-127 | a compound of the formula I | 5-ethyl-6-octyl-[1,2,4]triazolo[1,5-a]pyrimidin- |
| | | 7-ylamine |
| B-128 | a compound of the formula I | 5-ethyl-6-(3,5,5-trimethylhexyl)-[1,2,4]triazolo- |
| | | [1,5-a]pyrimidin-7-ylamine |
| B-129 | a compound of the formula I | 6-octyl-5-propyl-[1,2,4]triazolo[1,5-a]pyrimidin- |
| | | 7-ylamine |
| B-130 | a compound of the formula I | 5-methoxymethyl-6-octyl-[1,2,4]triazolo[1,5-a]- |
| | | pyrimidin-7-ylamine |
| B-131 | a compound of the formula I | 6-octyl-5-trifluoromethyl-[1,2,4]triazolo[1,5- |
| | | a]pyrimidin-7-ylamine |
| B-132 | a compound of the formula I | 5-trifluoromethyl-6-(3,5,5-trimethylhexyl)- |
| | | [1,2,4]triazolo[1,5-a]pyrimidin-7-ylamine |
| B-133 | a compound of the formula I | 2-butoxy-6-iodo-3-propylchromene-4-one |
| B-134 | a compound of the formula I | N,N-dimethyl-3-(3-bromo-6-fluoro-2-methyl- |
| | | indole-1-sulfonyl)-[1,2,4]triazole-1-sulfon- |
| | | amide |
| B-135 | a compound of the formula I | ferbam |
| B-136 | a compound of the formula I | mancozeb |
| B-137 | a compound of the formula I | maneb |
| B-138 | a compound of the formula I | metiram |
| B-139 | a compound of the formula I | metam |
| B-140 | a compound of the formula I | propineb |
| B-141 | a compound of the formula I | thiram |
| B-142 | a compound of the formula I | zineb |
| B-143 | a compound of the formula I | ziram |
| B-144 | a compound of the formula I | diethofencarb |
| B-145 | a compound of the formula I | flubenthiavalicarb |
| B-146 | a compound of the formula I | iprovalicarb |
| B-147 | a compound of the formula I | propamocarb |
| B-148 | a compound of the formula I | methyl 3-(4-chlorophenyl)-3-(2-isopropoxy- |
| | | carbonylamino-3-methylbutyrylamino)- |
| | | propionate |
| B-149 | a compound of the formula I | 4-fluorophenyl N-(1-(1-(4-cyanophenyl)- |
| | | ethanesulfonyl)but-2-yl)carbamate |
| B-150 | a compound of the formula I | dodine |
| B-151 | a compound of the formula I | iminoctadine |
| B-152 | a compound of the formula I | guazatine |
| B-153 | a compound of the formula I | kasugamycin |
| B-154 | a compound of the formula I | polyoxine |
| B-155 | a compound of the formula I | streptomycin |
| B-156 | a compound of the formula I | validamycin A |
| B-157 | a compound of the formula I | fentin salts |
| B-158 | a compound of the formula I | isoprothiolane |
| B-159 | a compound of the formula I | dithianon |
| B-160 | a compound of the formula I | edifenphos |
| B-161 | a compound of the formula I | fosetyl |
| B-162 | a compound of the formula I | fosetyl-aluminum |
| B-163 | a compound of the formula I | iprobenfos |
| B-164 | a compound of the formula I | pyrazophos |
| B-165 | a compound of the formula I | tolclofos-methyl |
| B-166 | a compound of the formula I | phosphorous acid and its salts |
| B-167 | a compound of the formula I | thiophanate methyl |
| B-168 | a compound of the formula I | chlorothalonil |
| B-169 | a compound of the formula I | dichlofluanid |
| B-170 | a compound of the formula I | tolylfluanid |
| B-171 | a compound of the formula I | flusulfamide |
| B-172 | a compound of the formula I | phthalide |
| B-173 | a compound of the formula I | hexachlorobenzene |
| B-174 | a compound of the formula I | pencycuron |
| B-175 | a compound of the formula I | quintozene |
| B-176 | a compound of the formula I | binapacryl |
| B-177 | a compound of the formula I | dinocap |
| B-178 | a compound of the formula I | dinobuton |
| B-179 | a compound of the formula I | Bordeaux mixture |
| B-180 | a compound of the formula I | copper acetate |
| B-181 | a compound of the formula I | copper hydroxide |
| B-182 | a compound of the formula I | copper oxychloride |
| B-183 | a compound of the formula I | basic copper sulfate |
| B-184 | a compound of the formula I | sulfur |
| B-185 | a compound of the formula I | spiroxamine |
| B-186 | a compound of the formula I | cyflufenamid |
| B-187 | a compound of the formula I | cymoxanil |
| B-188 | a compound of the formula I | metrafenone |

The active compounds II, mentioned above as component 2, their preparation and their action against harmful fungi are generally known (cf.: http://www.hclrss.demon.co.uk/index.html); they are commercially available. The compounds named according to IUPAC, their preparation and their fungicidal action are likewise known [cf. EP-A 226 917; EP-A 10 28 125; EP-A 10 35 122; EP-A 12 01 648; WO 98/46608; WO 99/24413; WO 03/14103; WO 03/053145; WO 03/066609; WO 04/049804 and WO 07/012,598].

Compositions of this invention may also contain other active ingredients, for example other pesticides such as insecticides and herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents usually are admixed with the agents according to the invention in a weight ratio of 1:100 to 100:1.

The following list of pesticides together with which the compounds according to the invention can be used, is intended to illustrate the possible combinations, but not to impose any limitation:

A.1. Organo(thio)phosphates: e.g. acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;

A.2. Carbamates: e.g. alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;

A.3. Pyrethroids: e.g. allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;

A.4. Growth regulators: a) chitin synthesis inhibitors: e.g. benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: e.g. halofenozide, methoxyfenozide, tebufenozide, azadirachtin; c) juvenoids: e.g. pyriproxyfen, methoprene, fenoxycarb; d) lipid biosynthesis inhibitors: e.g. spirodiclofen, spiromesifen or spirotetramat;

A.5. Nicotinic receptor agonists/antagonists compounds (nicotinoid insecticides or neonicotinoids): e.g. clothianidin, dinotefuran, imidacloprid, thiamethoxam, nitenpyram, acetamiprid, thiacloprid or the thiazol compound of formula P1

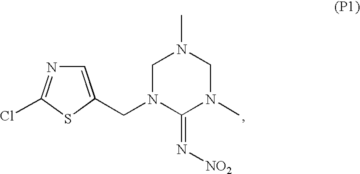

A.6. GABA antagonist compounds: e.g. acetoprole, endosulfan, ethiprole, fipronil, vaniliprole, pyrafluprole, pyriprole, 5-amino-3-(aminothiocarbonyl)-1-(2,6-dichloro-4-trifluoromethylphenyl)-4-(trifluoromethylsulfinyl)-pyrazole;

A.7. Macrocyclic lactone insecticides: abamectin, emamectin, milbemectin, lepimectin, spinosad,

A.8. Mitochondrial complex I electron transport inhibitors (METI I compounds): e.g. fenazaquin, pyridaben, tebufenpyrad, tolfenpyrad, flufenerim;

A.9. Mitochondrial complex II and/or complex Ill electron transport inhibitors (METI II and III compounds): e.g. acequinocyl, fluacyprim, hydramethylnon;

A.10. Uncoupler compounds: e.g. chlorfenapyr;

A.11. Oxidative phosphorylation inhibitor compounds: cyhexatin, diafenthiuron, fenbutatin oxide, propargite;

A.12. Moulting disruptor compounds: e.g. cyromazine;

A.13. Mixed function oxidase inhibitor compounds: e.g. piperonyl butoxide;

A.14. Sodium channel blocker compounds: e.g. indoxacarb, metaflumizone,

A.15. Various: benclothiaz, bifenazate, cartap, flonicamid, pyridalyl, pymetrozine, sulfur, thiocyclam, flubendiamide, cyenopyrafen, flupyrazofos, cyflumetofen, amidoflumet, compounds of the formula P2:

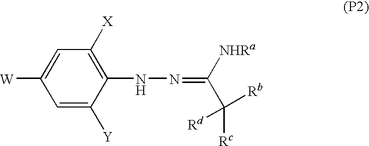

wherein X and Y are each independently halogen, in particular chlorine;
W is halogen or C₁-C₂-haloalkyl, in particular trifluoromethyl;
R^{a} is C₁-C₆-alkyl, C₂-C₆-alkenyl, C₂-C₆-alkynyl, C₁-C₄-alkoxy-C₁-C₄-alkyl or C₃-C₆-cycloalkyl each of which may be substituted with 1, 2, 3, 4 or 5 halogen atoms; in particular R^{a} is methyl or ethyl;
R^{b} and R^{c} are C₁-C₆-alkyl, in particular methyl, or may form together with the adjacent carbon atom a C₃-C₆-cycloalkyl moiety, in particular a cyclopropyl moiety, which may carry 1, 2 or 3 halogen atoms, examples including 2,2-dichlorocyclopropyl and 2,2-dibromocyclopropyl; and
R^{d} is hydrogen or C₁-C₆-alkyl, in particular hydrogen methyl or ethyl;
anthranilamide compounds of formula P3

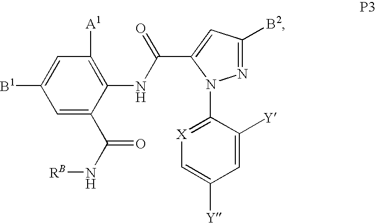

wherein A¹ is CH₃, Cl, Br, I, X is C—H, C—Cl, C—F or N, Y′ is F, Cl, or Br, Y″ is F, Cl, CF₃, B′ is hydrogen, Cl, Br, I, CN, B² is Cl, Br, CF₃, OCH₂CF₃, OCF₂H, and R^{B} is hydrogen, CH₃ or CH(CH₃)₂;
   and malononitrile compounds as described in JP 2002/284608, WO 02/89579, WO 02/90320, WO 02/90321, WO 04/06677, WO 04/20399 or JP 2004/99597.

Suitable pesticides compounds also include microorganisms such as Bacillus thuringiensis, Bacillus tenebrionis and Bacillus subtilis.

The aforementioned compositions are particularly useful for protecting plants against infestation of said pests and also for protecting plants against infections of phytopathogenic fungi or to combat these pests/fungi in infested/infected plants.

However, the compounds of formula I are also suitable for the treatment of seeds. Application to the seeds is carried out before sowing, either directly on the seeds or after having pregerminated the latter.

Compositions which are useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)
H Dustable powders (DP, DS)

Preferred FS formulations of compounds of formula I for seed treatment usually comprise from 0.5 to 80% of the active ingredient, from 0.05 to 5% of a wetter, from 0.5 to 15% of a dispersing agent, from 0.1 to 5% of a thickener, from 5 to 20% of an anti-freeze agent, from 0.1 to 2% of an anti-foam agent, from 1 to 20% of a pigment and/or a dye, from 0 to 15% of a sticker/adhesion agent, from 0 to 75% of a filler/vehicle, and from 0.01 to 1% of a preservative.

Suitable pigments or dyes for seed treatment formulations are pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Stickers/adhesion agents are added to improve the adhesion of the active materials on the seeds after treatment. Suitable adhesives are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers and copolymers derived from these polymers.

For use against ants, termites, wasps, flies, mosquitos, crickets, or cockroaches, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g. in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and its respective compositions can also be used in mosquito and fumigating coils, smoke cartridges, vaporizer plates or long-term vaporizers and also in moth papers, moth pads or other heat-independent vaporizer systems.

The compounds of formula I and its compositions can be used for protecting non-living material, in particular cellulose-based materials such as wooden materials e.g. trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

In the methods according to the invention the pests are controlled by contacting the target parasite/pest, its food supply, habitat, breeding ground or its locus with a pesticidally effective amount of at least one compounds I, or the N-oxide or salt thereof, or with a composition, containing a pesticidally effective amount of at least one compound I, or the N-oxide or salt thereof.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds I of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

The compounds I are employed by treating the fungi, pests or the plants, seeds, materials or the soil to be protected from fungal attack or pesticidal attack with a fungicidally or pesticidally effective amount of at least one active compound I, its N-oxide or salt. The application can be carried out both before and after the infection/infestation of the materials, plants or seeds by the fungi or pest.

When employed in plant protection, the amounts applied are, depending on the kind of effect desired, in the range of 0.1 g to 4000 g per hectare, desirably from 25 g to 600 g per hectare, more desirably from 50 g to 500 g per hectare.

In the treatment of seed, the application rates of the active compounds are generally from 0.001 g to 100 g per kg of seed, preferably from 0.01 g to 50 g per kg of seed, in particular from 0.01 g to 2 g per kg of seed.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in bait compositions, the typical content of active ingredient is from 0.001 weight % to 15 weight %, desirably from 0.001 weight % to 5% weight % of active compound.

For use in spray compositions, the content of active ingredient is from 0.001 to 80 weights %, preferably from 0.01 to 50 weight % and most preferably from 0.01 to 15 weight %.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

Under outdoor conditions, the active compound application rate for controlling pests is from 0.1 to 2.0, preferably from 0.2 to 1.0, kg/ha.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Adjuvants which can be used are in particular organic modified polysiloxanes such as Break Thru S 240®; alcohol alkoxylates such as Atplus 245®, Atplus MBA 1303®, Plurafac LF 300® and Lutensol ON 30®; EO/PO block polymers, z. B. Pluronic RPE 2035® and Genapol B®; alcohol ethoxylates such as Lutensol XP 80®; and dioctyl sulfosuccinate sodium such as Leophen RA®.

Compounds of formula I, their N-oxides and veterinarily acceptable salts as well as the compositions comprising them can also be used for controlling and preventing infestations and infections in animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I and compositions comprising them are suitable for systemic and/or non-systemic control of ecto- and/or endoparasites. They are active against all or some stages of development.

Administration can be carried out both prophylactically and therapeutically. Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds, their N-oxides and salts may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Generally it is favorable to apply solid formulations which release compounds of formula I in total amounts of 10 mg/kg to 300 mg/kg, preferably 20 mg/kg to 200 mg/kg. The active compounds can also be used as a mixture with synergists or with other active compounds which act against pathogenic endo- and ectoparasites.

In general, the compounds of formula I are applied in parasiticidally effective amount-meaning the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

### SYNTHESIS EXAMPLES

The procedures described in the synthesis examples below were used to prepare further compounds I by appropriate modification of the starting compounds. The compounds thus obtained are listed in the tables below, together with physical data.

### Example 1

### Preparation of 4-bromo-thiophene-2-sulfonic acid picolyl amide

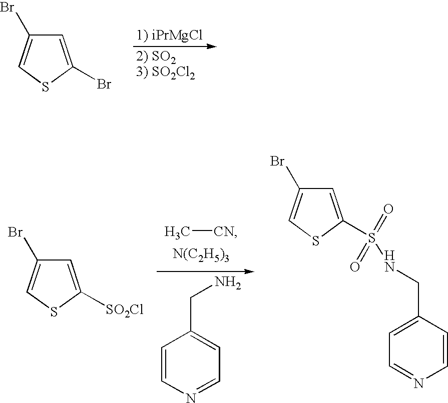

At 0° C. a solution isopropyl-magnesiumchloride (18% in tetrahydrofuran, 1.1 eq) was slowly added to 2,4-dibromothiophene (242 g, 1 mol) in tetrahydrofuran (1000 ml), thereby maintaining the temperature between 0 at 10° C. After Stirring for 1 hour at about 20° C., the solution was cooled to (−40)° C. SO₂ (200 g, 3 eq) was added while maintaining the temperature at (−40)° C. with intense cooling. After 30 minutes at this temperature, SO₂Cl₂ (150 g, 1.1 eq) was added carefully and the reaction mixture was warmed to 0° C. After 30 minutes, the mixture was warmed to about 20° C. and 10% aqueous HCl was added carefully. The crude reaction mixture was extracted with methyl-tert.-butyl ether (3×1000 ml). The combined organic phases were washed with saturated aqueous NaCl (600 ml) and dried over Na₂SO₄.

The solvent was removed and the crude sulfochloride was dissolved in acetonitrile (600 ml). Meanwhile, picolylamine (122 g, 1.1 eq) and triethylamine (114 g, 1.1 eq) were dissolved in methylcyanide (1400 ml) and cooled to 0° C. The crude sulfochloride in methylcyanide was added via dropping funnel maintaining the temperature below 10° C. The solution was warmed to about 20° C. and stirred overnight. The precipitated solid was filtered off and washed with 100 ml water. The desired product (193 g) was obtained as an off-white solid.

### Example 2

### Preparation of 5-(4-methoxyphenyl)-pyridine-2-sulfonic acid picolyl amide

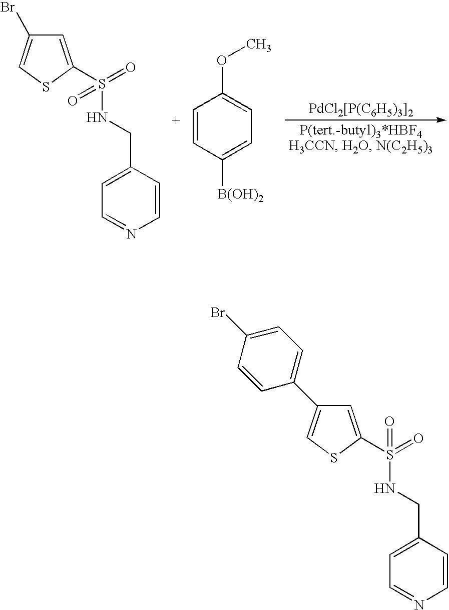

A solution of the thienylbromide (0.41 g, 1.2 mmol), boronic acid (0.22 g, 1.5 mmol), PdCl₂[P(C₆H₅)₃]₂ (0.03 g), P(tert.-butyl)₃*HBF₄ (0.020 g) and triethylamine (0.42 g) was dissolved in methylcyanide (5 ml) and water (2 ml). The reaction mixture was refluxed for 2 hours. After chromatography the title compound (0.23 g) was obtained as a off-white solid. Mp.: 150° C.

Compounds I listed in Table C and Table D were prepared in an analogous manner.

TABLE C

| | |
|---|---|
| | Ia |
| 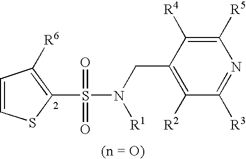 | |

| Compound | R¹ | R² | R³ | R⁴ | R⁵ | R⁶ | Phys. Data: m.p. |
|---|---|---|---|---|---|---|---|
| Ia.1 | H | H | H | H | H | p-(CH₂—C₂H₅)-phenyl | 115-117° C. |
| Ia.2 | H | H | H | H | H | p-C₂H₅-phenyl | 158-160° C. |
| Ia.3 | H | H | H | H | H | p-F-phenyl | 182-184° C. |
| Ia.4 | H | H | H | H | H | p-CH(CH₃)₂-phenyl | 142° C. |
| Ia.5 | H | H | H | H | H | p-Cl-phenyl | 140-148° C. |
| Ia.6 | H | H | H | H | H | m-Cl-phenyl | 175-177° C. |

m.p. melting point

TABLE D

| | |
|---|---|
| | Ib |
| 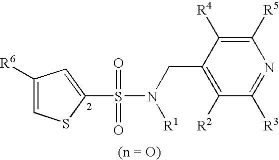 | |

| Compound | R¹ | R² | R³ | R⁴ | R⁵ | R⁶ | Phys. Data: m.p. |
|---|---|---|---|---|---|---|---|
| Ib.1 | H | H | H | H | H | p-C₂H₅-phenyl | 158-160° C. |
| Ib.2 | H | H | H | H | H | p-F-phenyl | 156-158° C. |
| Ib.3 | H | H | H | H | H | m-Cl-phenyl | 102° C. |
| Ib.4 | H | H | H | H | H | p-CF₃-phenyl | 158-160° C. |
| Ib.5 | H | H | H | H | H | p-CH(CH₃)₂-phenyl | 172° C. |
| Ib.6 | H | H | H | H | H | p-OCF₃-phenyl | 116-118° C. |
| Ib.7 | H | H | H | H | H | p-Cl-phenyl | 184-186° C. |
| Ib.8 | H | H | H | H | H | p-CH₂—C₂H₅)-phenyl | 152° C. |
| Ib.9 | H | H | H | H | H | p-(CO—CH₃)-phenyl | 182-184° C. |
| Ib.10 | H | H | H | H | H | p-(C(CH₃)═NOCH₃)-phenyl | 175-176° C. |
| Ib.11 | H | H | H | H | H | p-(C(CH₃)═NOC₂H₅)-phenyl | 212-215° C. |
| Ib.12 | H | H | H | H | H | m-Cl, p-(OCH₃)-phenyl | 140-145° C. |
| Ib.13 | H | H | H | H | H | m,p-(O—CH₂—O)-phenyl | 140-145° C. |
| Ib.14 | H | H | H | H | H | o-Cl-phenyl | 112-115° C. |
| Ib.15 | H | H | H | H | H | m-Cl, p-F-phenyl | 180-182° C. |
| Ib.16 | H | H | H | H | H | p-CN-phenyl | 210-213° C. |
| Ib.17 | H | H | H | H | H | m-CN-phenyl | 168-172° C. |
| Ib.18 | H | H | H | H | H | m-F, p-F-phenyl | 185° C. |
| Ib.19 | H | H | H | H | H | m-Cl, p-Cl-phenyl | 165-168° C. |
| Ib.20 | H | H | H | H | H | o-CH₃, p-F-phenyl | 156-157° C. |
| Ib.21 | H | H | H | H | H | p-CH₃-phenyl | 165° C. |
| Ib.22 | H | H | H | H | H | o-CH₃-phenyl | 134° C. |
| Ib.23 | H | H | H | H | H | m-CH₃-phenyl | 93° C. |
| Ib.24 | H | H | H | H | H | m-F-phenyl | |
| Ib.25 | H | H | H | H | H | o-F-phenyl | 107° C. |
| Ib.26 | H | H | H | H | H | m-CF₃-phenyl | 153° C. |
| Ib.27 | H | —CH═CH—CH═CH— | | H | H | p-OCF₃-phenyl | ¹H-NMR (in d⁶-di- |
| | | | | | | | methylsulfoxide): |
| | | | | | | | δ [ppm] = 8.8 |
| | | | | | | | (m, 1H), 8.1-7.9 |
| | | | | | | | (m, 4H), 7.8 |
| | | | | | | | (m, 2H), 7.7-7.5 |
| | | | | | | | (m, 4H), 7.4 |
| | | | | | | | (m, 1H), 7.3 |
| | | | | | | | (m, 2H), 4.6 (d, 2H) |
| Ib.28 | H | H | H | H | H | p-OCH₃-phenyl | 150° C. |
| Ib.29 | H | H | H | H | H | m-OCH₃-phenyl | 63° C. |
| Ib.30 | H | H | H | H | H | o-OCH₃-phenyl | 65° C. |
| Ib.31 | H | H | H | H | H | o-(CO—NH₂)-phenyl | 187° C. |
| Ib.32 | H | H | H | H | H | o-CF₃-phenyl | |

| | | | | | | |
|---|---|---|---|---|---|---|
| Ib.33 | H | —CH═CH—CH═CH— | H | H | o-Cl, p-Cl-phenyl | 155-156° C. |
| Ib.34 | H | —CH═CH—CH═CH— | H | H | o-CF₃, p-CF₃-phenyl | 160-161° C. |

### Examples of the Action Against Harmful Fungi

The fungicidal action of the compounds of the formula I was demonstrated by the following experiments:

The spray soultions were prepared in several steps:
a stock solution was prepared: a mixture of acetone and/or dimethylsulfoxide and the wetting agent/emulsifier Uniperol® EL, which is based on ethoxylated alkylphenoles, in a relation (volume) solvent-emulsifier of 99 to 1 was added to 25 mg of the active compound to give a total of 10 ml.
Water was then added to total volume of 100 ml.
This stock solution was diluted with the described solvent-emulsifier-water mixture to the given concentration.

The active compounds were formulated separately or together as a stock solution with 0.25% by weight of active compound in acetone or dimethylsulfoxide. 1% by weight of the emulsifier Uniperol® EL (wetting agent having emulsifying and dispersing action based on ethoxylated alkylphenols) was added to this solution and diluted with water to the desired concentration.

### Use Example 1

### Preventive Fungicidal Control of Early Blight on Tomatoes Caused by Alternaria Solani

Young seedlings of tomato plants were grown in pots. These plants were sprayed to run-off with an aqueous suspension, containing the concentration of active ingredient stated below. The next day, the treated plants were inoculated with an aqueous spore suspension of Alternaria solani containing 0.17×10⁶ spores per ml. Then the trial plants were immediately transferred to a humid chamber. After 5 days at 20 and 22° C. and a relative humidity close to 100%, the extent of fungal attack on the leaves was visually assessed as % diseased leaf area.

In this test the plants which have been treated with 250 ppm of the active compound Ib.1, Ib.3, Ib.4, Ib.5, Ib.6, Ib.10, Ib.11, Ib.13, Ib.14, Ib.17, Ib.21, Ib.23 and Ib.26, respectively, showed an infection of not more than 20%, whereas the untreated plants were 90% infected.

### Use Example 2

### Control of Late Blight on Tomatoes Caused by Phytophthora infestans, Protective Treatment

Young seedlings of tomato plants were grown in pots. The plants were sprayed to runoff with an aqueous suspension containing the concentration of active ingredient stated below. The next day, the treated plants were inoculated with an aqueous suspension of sporangia of Phytophthora infestans. After inoculation, the trial plants were immediately transferred to a humid chamber. After six days at 18 to 20° C. and a relative humidity close to 100%, the extent of fungal attack on the leaves was visually assessed as % diseased leaf area.

In this test the plants which have been treated with 250 ppm of the active compound Ib.4, Ib.10, Ib.12, Ib.13, Ib.14, Ib.21 and Ib.23, respectively, showed an infection of not more than 20%, whereas the untreated plants were 90% infected.

### Use Example 3

### Protective Control of Powdery Mildew on Cocumber Caused by Sphaerotheca Fuliginea

Cucumber seedlings were grown in pots to the cotyledon stage. The plants were sprayed to run-off with an aqueous suspension, containing the concentration of active ingredient as given below. The next day the treated plants were inoculated with an aqueous spore suspension of cucumber powdery mildew (Sphaerotheca fuliginea). Then the trial plants were cultivated in a greenhouse at temperatures between 20 and 24° C. and a relative humidity between 60 and 80%. After 8 days the extent of fungal attack on the leaves was visually assessed as % diseased leaf area.

The action of the compounds of the formula I against harmful pests was demonstrated by the following experiments:

1. Activity against Boll weevil (Anthonomus grandis)

The active compounds were formulated in 1:3 dimethylsulfoxide/water. 10 to 15 eggs were placed into microtiterplates filled with 2% agar-agar in water and 300 ppm formaline. The eggs were sprayed with 20 μl of the test solution, the plates were sealed with pierced foils and kept at 24-26° C. and 75-85% humidity with a day/night cycle for 3 to 5 days. Mortality was assessed on the basis of the remaining unhatched eggs or larvae on the agar surface and/or quantity and depth of the digging channels caused by the hatched larvae. Tests were replicated 2 times.

In this test the eggs which have been treated with 2500 ppm of the active compounds Ib.7, Ib.13, Ib.18, Ib.19, Ib.33 and Ib.34, respectively, showed a mortality of at least 75%.

2. Activity against Mediterranean fruitfly (Ceratitis capitata)

The active compounds were formulated in 1:3 Dimethylsulfoxide/water. 50 to 80 eggs were placed into microtiterplates filled with 0.5% agar-agar and 14% diet in water. The eggs were sprayed with 5 μl of the test solution, the plates were sealed with pierced foils and kept at 27-29° C. and 75-85% humidity under fluorescent light for 6 days. Mortality was assessed on the basis of the agility of the hatched larvae. Tests were replicated 2 times.

In this test the eggs which have been treated with 2500 ppm of the active compounds Ia.6, Ib.6 and Ib.11, respectively, showed a mortality of at least 75%.

3. Activity against Tobacco budworm (Heliothis virescens)

The active compounds were formulated in 1:3 dimethylsulfoxide/water. 15 to 25 eggs were placed into microtiterplates filled with diet. The eggs were sprayed with 10 μl of the test solution, the plates were sealed with pierced foils and kept at 27-29° C. and 75-85% humidity under fluorescent light for 6 days. Mortality was assessed on the basis of the agility and of comparative feeding of the hatched larvae. Tests were replicated 2 times. In this test the eggs which have been treated with 2500 ppm of the active compounds Ib.7, Ib.19, Ib.20 and Ib.33, respectively, showed a mortality of at least 75%.

4. Activity against Vetch aphid (Megoura viciae)

The active compounds were formulated in 1:3 Dimethylsulfoxide/water. Bean leaf disks were placed into microtiterplates filled with 0.8% agar-agar and 2.5 ppm OPUS®. The leaf disks were sprayed with 2.5 μl of the test solution and 5 to 8 adult aphids were placed into the microtiterplates which were then closed and kept at 22-24° C. and 35-45% under fluorescent light for 6 days. Mortality was assessed on the basis of vital, reproduced aphids. Tests were replicated 2 times.

We claim:

1-24. (canceled)

25. A compound of formula I 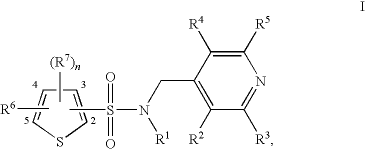
wherein:
R¹ is hydrogen, C₁-C₄-alkyl, C₁-C₄-alkoxy, cyano-C₁-C₄-alkyl, C₁-C₄-haloalkyl, C₁-C₄-alkoxy-C₁-C₄-alkyl, C₁-C₄-haloalkoxy-C₁-C₄-alkyl, di(C₁-C₄-alkyl)-amino-C₁-C₄-alkyl, C₃-C₆-cycloalkyl-C₁-C₄-alkyl, C₃-C₆-halocycloalkyl-C₁-C₄-alkyl, (C₁-C₄-alkyl)carbonyl, (C₁-C₄-alkoxy)carbonyl, C₂-C₄-alkenyl, cyano-C₂-C₄-alkenyl, C₂-C₄-haloalkenyl, C₁-C₄-alkoxy-C₂-C₄-alkenyl, C₁-C₄-halo-alkoxy-C₂-C₄-alkenyl, (C₁-C₄-alkyl)carbonyl-C₂-C₄-alkenyl, (C₁-C₄-alkoxy)-carbonyl-C₂-C₄-alkenyl, di(C₁-C₄-alkyl)amino-C₂-C₄-alkenyl, C₂-C₄-alkynyl, C₂-C₄-haloalkynyl, C₁-C₄-alkyl-C₂-C₄-alkynyl, C₁-C₄-haloalkyl-C₂-C₄-alkynyl, C₁-C₄-alkoxy-C₂-C₄-alkynyl, di(C₁-C₄-alkyl)amino, or benzyl which may bear at the phenyl ring a cyano, halogen, C₁-C₄-alkyl, C₁-C₄-haloalkyl, C₁-C₄-alkoxy, C₁-C₄-haloalkoxy, (C₁-C₄-alkyl)carbonyl, (C₁-C₄-alkoxy)carbonyl or di(C₁-C₄-alkyl)amino radical;
R², R³ independently of one another are selected from hydrogen, thiol, amino, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkyl-sulfinyl, C₁-C₄-alkylsulfonyl, C₁-C₄-haloalkylsulfonyl, (C₁-C₄-alkyl)amino, di(C₁-C₄-alkyl)amino, tri-C₁-C₄-alkylsilyl, C₂-C₄-alkenyl, C₂-C₄-alkynyl or
5- or 6-membered heterocyclyl ring containing one nitrogen atom and optionally a second heteroatom selected from oxygen, sulfur, NH or N(C₁-C₄-alkyl);
R⁴, R⁵ independently of one another are selected from hydrogen, halogen, C₁-C₄-alkyl, C₁-C₄-haloalkyl, C₁-C₄-alkoxy or C₁-C₄-haloalkoxy; or
R² and R³ together with the carbon atoms to which they are attached, may form a condensed phenyl, cyclopentyl, cyclohexyl or 5- or 6-membered heterocyclyl ring containing one to three heteroatoms selected from the group consisting of 2 nitrogen, 1 oxygen and 1 sulfur atoms,
it being possible for all these rings to carry one or two groups R⁸ and/or R⁹,
R⁸, R⁹ independently of one another are halogen, C₁-C₄-alkyl, C₁-C₄-haloalkyl, C₁-C₄-alkoxy or C₁-C₄-haloalkoxy;
R⁶ is halogen, cyano, nitro, C₁-C₆-alkyl, C₁-C₆-alkoxy, C₁-C₆-haloalkoxy, (C₁-C₄-alkyl)carbonyl, (C₁-C₄-alkoxy)carbonyl, —C(R¹⁰)═NOR¹¹, (C₁-C₄-alkyl)-aminocarbonyl, di(C₁-C₄-alkyl)aminocarbonyl, phenyl or phenoxy, where the ring in the last two mentioned radicals may carry one, two or three groups R¹²;
R⁷ cyano, formyl, halogen, C₁-C₄-alkyl, C₁-C₄-haloalkyl, C₁-C₄-alkoxy, C₁-C₄-haloalkoxy, C₁-C₄-alkoxy-C₁-C₄-alkyl, (C₁-C₄-alkoxy)carbonyl, aminocarbonyl, C₁-C₄-alkylaminocarbonyl or di(C₁-C₄-alkyl)aminocarbonyl;
n is zero or one;
or
R⁶ and R⁷ together with the carbon atoms to which they are attached, may form a condensed phenyl ring, it being possible for the phenyl ring to carry one C₁-C₄-alkyl group;
R¹⁰ is hydrogen, C₁-C₄-alkyl, C₁-C₄-haloalkyl, C₁-C₄-alkoxy-C₁-C₄-alkyl, C₁-C₄-haloalkoxy-C₁-C₄-alkyl, phenyl which may bear a cyano, halogen, C₁-C₄-alkoxy or C₁-C₄-haloalkoxy radical, or benzyl which may bear a cyano, halogen or C₁-C₄-alkyl radical;
R¹¹ is C₁-C₆-alkyl, benzyl, C₂-C₄-alkenyl, C₁-C₄-haloalkyl, C₂-C₄-haloalkenyl, C₂-C₄-alkynyl or C₂-C₄-haloalkynyl;
R¹² is nitro, cyano, halogen, C₁-C₄-alkyl, C₁-C₄-haloalkyl, C₁-C₄-alkoxy, C₁-C₄-haloalkoxy, (C₁-C₄-alkoxy)carbonyl, C₁-C₄-alkylthio, C₁-C₄-haloalkylthio, C₁-C₄-alkylsulfonyl, C₁-C₄-haloalkylsulfonyl, (C₁-C₄-alkyl)amino, di(C₁-C₄-alkyl)amino, tri(C₁-C₄-alkyl)silyl, —CH═NO(C₁-C₄-alkyl), —C(C₁-C₄-alkyl)═NO(C₁-C₄-alkyl), C₂-C₄-alkenyl or C₃-C₄-alkinyl;
or two radicals R¹² may form a C₃-C₄-alkylene or C₄-alkenylene chain which, together with two adjacent ring members of the aryl ring to which it is attached, forms a ring which may be substituted by one to 3 groups R¹³;
R¹³ is halogen, cyano, nitro, C₁-C₈-alkyl, C₁-haloalkyl, C₁-C₈-alkoxy, C₁-C₈-haloalkoxy, (C₁-C₄-alkyl)carbonyl, (C₁-C₄-alkoxy)carbonyl, —C(R¹⁴)═NOR¹⁵, (C₁-C₄-alkyl)aminocarbonyl, di(C₁-C₄-alkyl)aminocarbonyl, phenyl or phenoxy, where the ring in the last two mentioned radicals may carry one, two or three groups R¹⁶;
R¹⁴ is hydrogen, C₁-C₄-alkyl, C₁-C₄-haloalkyl, C₁-C₄-alkoxy-C₁-C₄-alkyl, C₁-C₄-haloalkoxy-C₁-C₄-alkyl, phenyl which may bear a cyano, halogen, C₁-C₄-alkoxy or C₁-C₄-haloalkoxy radical, or benzyl which may bear a cyano, halogen or C₁-C₄-alkyl radical;
R¹⁵ is C₁-C₆-alkyl, benzyl, C₂-C₄-alkenyl, C₁-C₄-haloalkyl, C₂-C₄-haloalkenyl, C₂-C₄-alkynyl or C₂-C₄-haloalkynyl;
R¹⁶ is halogen, C₁-C₄-alkyl, C₁-C₄-alkoxy, C₁-haloalkyl or C₁-haloalkoxy;
and the N-oxides and the agriculturally acceptable salts and the veterinarily acceptable salts of the compounds I,
with the proviso that if the thiophene ring is bonded to the sulfonyl group via position 2, R⁶ cannot be at position 5.

26. The compound of claim 25, wherein R², R³, R⁴ and R⁵ are hydrogen.

27. The compound of claim 25, wherein R² and R³, independently of one another, are selected from the group consisting of hydrogen, amino, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, (C₁-C₄-alkyl)amino, di(C₁-C₄-alkyl)amino, C₂-C₄-alkenyl, C₂-C₄-alkynyl and tri-C₁-C₄-alkylsilyl, and R⁴ and R⁵ are hydrogen, wherein at least one of the radicals R² and R³ is different from hydrogen.

28. The compound of claim 25, wherein R² and R³, together with the carbon atoms to which they are attached, form a fused benzene ring, it being possible for the fused benzene ring to carry one or two radicals R⁸ and/or R⁹, and wherein R⁴ and R⁵ are hydrogen.

29. The compound of claim 25, wherein R¹ is hydrogen, C₁-C₄-alkyl, C₃-C₄-alkenyl, C₃-C₄-alkynyl or benzyl.

30. The compound of claim 25, wherein R⁶ is phenyl, which may carry 1, 2 or 3 radicals R¹².

31. The compound of claim 1, having formula Ia 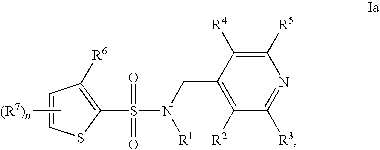
and the N-oxides and the agriculturally acceptable salts thereof.

32. The compound of claim 1, having the formula Ib 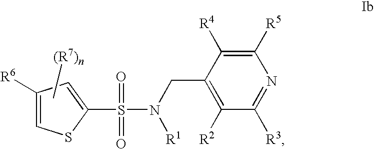
and the N-oxides and the agriculturally acceptable salts thereof.

33. The compound of claim 1, having the formula Ic 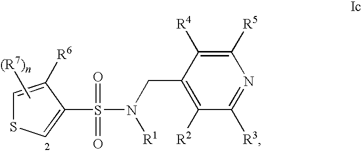
and the N-oxides and the agriculturally acceptable salts thereof.

34. The compound of claim 1, having the formula Id 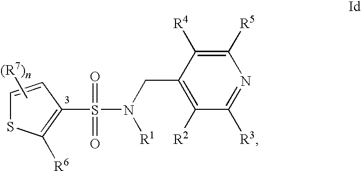
and the N-oxides and the agriculturally acceptable salts thereof.

35. The compound of claim 1, having the formula Ie 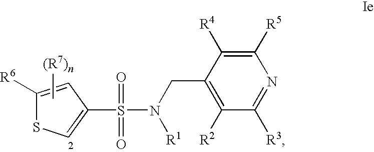
and the N-oxides and the agriculturally acceptable salts thereof.

36. A process for the preparation of a compound of formula I of claim 1, comprising reacting a compound of formula II 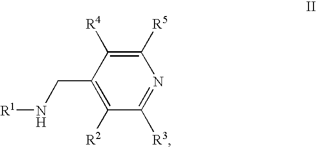
wherein R¹ to R⁵ are as defined in claim 1, under basic conditions with a compound of formula III 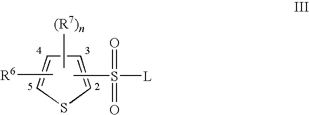
wherein L is hydroxy or halogen.

37. An agricultural composition comprising a solid or liquid carrier and at least one compound of formula I of claim 1 or an N-oxide or an agriculturally acceptable salt thereof.

38. A process for the treatment of phytopathogenic harmful fungi comprising, treating the fungi or the materials, plants, the soil or seeds to be protected against fungal attack, with an effective amount of at least one compound of the formula I of claim 1 or an N-oxide or an agriculturally acceptable salt thereof.

39. A method for combating arthropodal pests comprising, contacting said pests, their habitat, breeding ground, food supply, plant, seed, soil, area, material or environment in which the arthropodal pests are growing or may grow, or the materials, plants, seeds, soils, surfaces or spaces to be protected from an attack of or infestation by said pests, with a pesticidally effective amount of at least one compound of formula I of claim 1, an N-oxide, an agriculturally acceptable salt or a veterinarily acceptable salt thereof, or with a composition comprising at least one compound of formula I, an N-oxide, an agriculturally acceptable salt or a veterinarily acceptable salt thereof.

40. The method of claim 39, wherein said pests are insects.

41. The method of claim 39, wherein said pests are arachnids.

42. A method for protecting crops from attack or infestation by arthropodal pests, comprising contacting a crop with a pesticidally effective amount of at least one compound of formula I of claim 1 or an N-oxide or an agriculturally acceptable salt thereof.

43. A method for protecting seed from infestation by arthropodal pests and of a seedlings' roots and shoots from infestation by arthropodal pests comprising contacting said seed or said seedlings' roots and shoots with a pesticidally effective amount of at least one compound of formula I of claim 1, or an N-oxide or an agriculturally acceptable salt thereof.

44. A method for protecting non-living materials from attack or infestation by arthropodal pests, comprising contacting said non-living material with a pesticidally effective amount of at least one compound of formula I of claim 1, or an N-oxide or an agriculturally acceptable salt thereof.

45. Seed comprising a compound of formula I of claim 1, or an N-oxide or an agriculturally acceptable salt thereof, in an amount of from 0.1 g to 10 kg per 100 kg of seed.
